(19) United States
(12) Patent Application Publication
(10) Pub. No.: US 2024/0401119 A1
(43) Pub. Date: Dec. 5, 2024

(54) CELLULAR STAINING PROBES FOR ANALYTE DETECTION

(71) Applicant: Waypoint Bio, Inc., New York, NY (US)

(72) Inventors: Xinchen Wang, New York, NY (US); David Vincent Phizicky, Jersey City, NJ (US)

(21) Appl. No.: 18/797,973

(22) Filed: Aug. 8, 2024

Related U.S. Application Data
(63) Continuation of application No. PCT/US2023/012792, filed on Feb. 10, 2023.
(60) Provisional application No. 63/309,418, filed on Feb. 11, 2022.

(51) Int. Cl. C12Q 1/6841; C12Q 1/6876

(52) U.S. Cl. CPC C12Q 1/6841; C12Q 1/6876; C12Q 2600/16

(57) ABSTRACT

The present disclosure relates to, in part, cleavable probes for detection of multiple analytes in a single sample and methods of use thereof. Specifically, probes comprising a binding agent linked to a detectable label by a cleavable linker (for example, a linker comprising a disulfide bond) can be used to detect analytes, then cleaved to remove the label, thus allowing further probing and detection of additional analytes.

### CROSS-REFERENCE TO RELATED APPLICATIONS

This application is a Continuation of International Application No. PCT/US2023/012792, filed on Feb. 10, 2023, which claims the benefit of and priority to U.S. Provisional Patent Application No. 63/309,418, filed Feb. 11, 2022, the disclosures of each of which are hereby incorporated by reference in their entireties for all purposes.

### FIELD OF THE INVENTION

The invention relates generally to methods and compositions for detecting the presence of one or more analytes in a sample.

### BACKGROUND

Fluorescence microscopy is one of the most important, pervasive and powerful imaging modalities in biomedical research. The spatial resolution of modern fluorescence microscopy has been improved to such a point that even sub-diffraction limited resolution is routinely possible. However, fluorescence imaging is often limited by the number of fluorophores one can detect with distinct excitation and emission spectra. For example, many imaging experiments are done using 4 different probes that detect one or more distinct analytes, and these 4 probes are bound directly or indirectly to different fluorophores represented by different colors (e.g., blue, green, orange, or red). Adding in more probes to detect other analytes often involves using non-standard fluorophores (e.g., near infrared fluorophores such as Alexa Fluor 750, which cannot be imaged on many microscopes) or fluorophores with a high degree of spectral overlap, which results in signal from specific probe-analyte pairs being difficult to ascertain (e.g., using Alexa Fluor 594 in addition to Alexa Fluor 555 and Alexa Fluor 647). Although these approaches can increase the number of probe-analyte pairs analyzed in a fluorescence imaging experiment, there are increasing costs with more fluorophores in terms of equipment or data processing, and the number of probe-analyte pairs is still limited.

Although cleavable fluorophores have been used for iterative rounds of imaging for nucleic acid-based probes, they have not been applied to probes that detect large cellular structures or organelles. The current inability to image many cellular structures and/or organelles in the same sample, or inability to image both cellular structures & organelles alongside other cellular features (e.g., nucleic acids, proteins and/or metabolites) is a major roadblock for the comprehensive characterization and understanding of biology.

### SUMMARY OF THE INVENTION

The present disclosure relates, in general, to methods and compositions for detecting analytes in a sample.

Disclosed herein, in certain embodiments, is a method for detecting the presence or absence, amount, location, morphology and/or spatial patterning of at least a first and a second analyte in a sample, the method comprising: (i) contacting the sample with a first probe comprising (a) a first binding agent capable of binding to the first analyte, and (b) a first detectable label, wherein the first binding agent and the first detectable label are conjugated by a first cleavable linker, under conditions to permit the first probe to bind the first analyte if the first analyte is present in the sample; (ii) detecting the first detectable label which, if present, is indicative of the presence or absence, amount, location, morphology and/or spatial patterning of the first analyte in the sample; (iii) cleaving the first cleavable linker in the first probe, thereby removing the first detectable label from the sample; (iv) contacting the sample with a second probe comprising (a) a second binding agent capable of binding to the second analyte, and (b) a second detectable label, optionally wherein the second binding agent and the second detectable label are conjugated by a second cleavable linker, under conditions to permit the second probe to bind the second analyte if the second analyte is present in the sample; and (v) detecting the second detectable label which, if present, is indicative of the presence or absence, amount, location, morphology and/or spatial patterning of the second analyte in the sample.

In some embodiments, the first cleavable linker and the second cleavable linker are each independently selected from the group consisting of a chemically cleavable linker, a photo-cleavable linker, and an enzymatically cleavable linker. In some embodiments, the first cleavable linker or the second cleavable linker is a chemically cleavable linker, or the first cleavable linker and the second cleavable linker are both a chemically cleavable linker. In some embodiments, the first cleavable linker or the second cleavable comprises a disulfide bond, or the first cleavable linker and the second cleavable linker comprise a disulfide bond.

In some embodiments, step (iii) comprises cleaving the first cleavable linker by contacting the sample with a first reducing agent. In some embodiments, the first reducing agent is selected from the group consisting of dithiothreitol (DTT), tris(2-carboxyethyl) phosphine (TCEP), and β-mercaptoethanol (BME).

In some embodiments of any of the foregoing methods, the first and second detectable labels are the same.

In some embodiments of any of the foregoing methods, the method detects the presence or absence, amount, location, morphology and/or spatial patterning of at least 50 analytes, at least 40 analytes, at least 30 analytes, at least 20 analytes, at least 15 analytes, at least 10 analytes, at least 9 analytes, at least 8 analytes, at least 7 analytes, at least 6 analytes, at least 5 analytes, at least 4 analytes, at least 3 analytes, or at least 2 analytes.

In some embodiments of any of the foregoing methods, the first or second binding agent is a protein. In some embodiments, the first or second binding agent is a lectin. In some embodiments, the first or second binding agent is Concanavalin A (ConA), wheat germ agglutinin (WGA), Isolectin GS-IB4, Lectin GS-II, or PNA lectin. In some embodiments, the first or second binding agent is ConA. In some embodiments, the first or second binding agent is WGA. In some embodiments, the first or second binding agent is a small molecule. In some embodiments, the first or second binding agent is not a nucleic acid. In some embodiments, the first and second detectable labels produce the same or a similar detectable signal.

In some embodiments of any of the foregoing methods, the first or second detectable label is a fluorophore. In some embodiments, the fluorophore is selected from the group consisting of an Alexa Fluor fluorophore, an ATTO dye, a cyanine dye, fluorescein, a fluorescent protein (e.g., GFP), a rhodamine dye, and a quenching agent (e.g., a Black Hole Quencher or an Iowa Black Quencher). In some embodiments, the fluorophore is selected from Alexa Fluor 488, Alexa Fluor 555, Alexa Fluor 568, Alexa Fluor 594, Alexa Fluor 633, Alexa Fluor 647, and Alexa Fluor 750. In some embodiments, the first and second detectable labels are fluorophores that have substantially overlapping excitation and/or emission spectra.

In some embodiments of any of the foregoing methods, step (i) comprises contacting the sample with at least 2 probes, at least 3 probes, at least 4 probes, at least 5 probes, at least 6 probes, at least 7 probes, at least 8 probes, at least 9 probes, or at least 10 probes. In some embodiments of any of the foregoing methods, step (iv) comprises contacting the sample with at least 2 probes, at least 3 probes, at least 4 probes, at least 5 probes, at least 6 probes, at least 7 probes, at least 8 probes, at least 9 probes, or at least 10 probes.

In some embodiments of any of the foregoing methods, the method further comprises (vi) cleaving the second cleavable linker in the probe in contact with the sample, thereby removing the second detectable label from the sample. In some embodiments, step (vi) comprises cleaving the second cleavable linker by contacting the sample with a second reducing agent. In some embodiments, the second reducing agent is selected from DTT, TCEP, and BME. In some embodiments, the first reducing agent and the second reducing agent are the same.

In some embodiments, the method further comprises: (vii) contacting the sample with a further probe comprising (a) a further binding agent capable of binding to a further analyte, and (b) a further detectable label, optionally wherein the further binding agent and the further detectable label are conjugated by a further cleavable linker, under conditions to permit the further probe to bind the further analyte if the further analyte is present in the sample; and (viii) detecting the further detectable label which, if present, is indicative of the presence, amount, location, morphology and/or spatial patterning of the further analyte in the sample.

In some embodiments, the method further comprises one or more iterations of: (ix) cleaving the cleavable linker in the probe in contact with the sample, thereby removing the detectable label from the sample, and subsequently repeating step (vii) and step (viii).

In some embodiments, the method comprises 2 or more, 3 or more, 4 or more, 5 or more, 6 or more, 7 or more, 8 or more, 9 or more, 10 or more, 11 or more, 12 or more, 13 or more, 14 or more, or 15 or more iterations of steps (ix), vii, and (viii).

In some embodiments, the further cleavable linker is selected from the group consisting of a chemically cleavable linker, a photo-cleavable linker, and an enzymatically cleavable linker. In some embodiments, the further cleavable linker comprises a disulfide bond. In some embodiments, step (ix) comprises cleaving the chemical linker by contacting the sample with a further reducing agent. In some embodiments, the further reducing agent is selected from the group consisting of DTT, TCEP, and BME. In some embodiments, the first, second, and further reducing agents are the same. In some embodiments, the first, second, and further detectable labels are the same.

In some embodiments, the further probe in each of the iterations of steps (ix), (vii), and (viii) comprises a different binding agent.

In some embodiments, the further binding agent is a protein. In some embodiments, the further binding agent is a lectin. In some embodiments, the further binding agent is Concanavalin A (ConA), wheat germ agglutinin (WGA), Isolectin GS-IB4, Lectin GS-II, or PNA lectin. In some embodiments, the first and/or second binding agent is ConA. In some embodiments, the first and/or second binding agent is WGA. In some embodiments, the further binding agent is a small molecule. In some embodiments, the further binding agent is not a nucleic acid. In some embodiments, the first, second, and further detectable labels produce the same or a similar detectable signal.

In some embodiments, the further detectable label is a fluorophore. In some embodiments, the further detectable label is selected from the group consisting of an Alexa Fluor fluorophore, an ATTO dye, a cyanine dye, fluorescein, a fluorescent protein (e.g., GFP), a rhodamine dye, and a quenching agent (e.g., a Black Hole Quencher or an Iowa Black Quencher). In some embodiments, the further detectable label is selected from Alexa Fluor 488, Alexa Fluor 555, Alexa Fluor 568, Alexa Fluor 594, Alexa Fluor 633, Alexa Fluor 647, and Alexa Fluor 750. In some embodiments, the first, second, and further detectable labels are fluorophores that have substantially overlapping excitation and/or emissions spectra.

In some embodiments, step (vii) comprises contacting the sample with at least 2 probes, at least 3 probes, at least 4 probes, at least 5 probes, at least 6 probes, at least 7 probes, at least 8 probes, at least 9 probes, or at least 10 probes.

In some embodiments of any of the foregoing methods, the sample is selected from a tissue sample, a liquid sample, and a cell sample. In some embodiments, the sample is a cell sample selected from a two-dimensional cell culture sample, a three-dimensional cell culture sample, a suspension cell culture sample, an organoid sample, a heterogeneous cell culture sample, and a patient-derived cell sample. In some embodiments, the first, second, and further analytes are each independently selected from the group consisting of a cell, organelle, protein, peptide, carbohydrate, glycoprotein, glycopeptide, glycolipid, lipid, lipoprotein, nucleic acid, and nucleoprotein. In some embodiments, the first, second, and further analytes are each independently selected from the group consisting of an endoplasmic reticulum, a plasma membrane, a Golgi body, a microtubule, an actin filament, a sarcomere, and a collagen fibril.

Also disclosed herein, in certain embodiments, is a method for detecting the presence or absence, amount, location, morphology and/or spatial patterning of at least a first and a second analyte in a sample, wherein the first analyte is the endoplasmic reticulum (ER) and/or a protein, carbohydrate, glycoprotein, or glycolipid enriched in the ER, the method comprising: (i) contacting the sample with a first probe comprising (a) ConA, and (b) a first fluorophore, wherein ConA and the first fluorophore are conjugated by a first cleavable linker comprising a disulfide bond, under conditions to permit ConA to bind the ER and/or the protein, carbohydrate, glycoprotein, or glycolipid enriched in the ER; (ii) detecting the first fluorophore; (iii) cleaving the disulfide bond in the first probe by addition of a reducing agent, thereby removing the first fluorophore from the sample; (iv) contacting the sample with a second probe comprising (a) a binding agent capable of binding to the second analyte, and (b) a second fluorophore, optionally wherein the binding agent and the second fluorophore are conjugated by a second cleavable linker comprising a disulfide bond; and (v) detecting the second fluorophore which, if present, is indicative of the presence, amount, location, morphology and/or spatial patterning of the second analyte in the sample.

In some embodiments, the reducing agent is selected from the group consisting of DTT, TCEP, and BME. In some embodiments, the binding agent is a protein. In some embodiments, the binding agent is a lectin. In some embodiments, the binding agent is WGA. In some embodiments, the binding agent is a small molecule. In some embodiments, the binding agent is not a nucleic acid. In some embodiments, the first and second fluorophores produce the same or a similar detectable signal. In some embodiments, the first and second fluorophore are independently selected from the group consisting of an Alexa Fluor fluorophore, an ATTO dye, a cyanine dye, fluorescein, a fluorescent protein (e.g., GFP), a rhodamine dye, and a quenching agent (e.g., a Black Hole Quencher or an Iowa Black Quencher). In some embodiments, the first and second fluorophore are independently selected from the group consisting of Alexa Fluor 488, Alexa Fluor 555, Alexa Fluor 568, Alexa Fluor 594, Alexa Fluor 633, Alexa Fluor 647, and Alexa Fluor 750. In some embodiments, the first and second detectable fluorophores have substantially overlapping excitation and/or emission spectra. In some embodiments, the method further comprises: (vi) cleaving the second cleavable linker in the probe in contact with the sample, thereby removing the second fluorophore from the sample. In some embodiments, the second reducing agent is selected from the group consisting of DTT, TCEP, and BME.

Also disclosed herein, in certain embodiments, is a method for detecting the presence or absence, amount, location, morphology and/or spatial patterning of at least a first, a second, and a third analyte in a sample, wherein the first analyte is a plasma membrane and/or a protein, carbohydrate, glycoprotein, or glycolipid enriched in the plasma membrane and the second analyte is a Golgi body and/or a protein, carbohydrate, glycoprotein, or glycolipid enriched in the Golgi body, the method comprising: (i) contacting the sample with a first probe comprising (a) WGA, and (b) a first fluorophore, wherein WGA and the first fluorophore are conjugated by a first cleavable linker comprising a disulfide bond, under conditions to permit WGA to bind the plasma membrane and/or the protein, carbohydrate, glycoprotein, or glycolipid enriched in the plasma membrane and the Golgi body and/or the protein, carbohydrate, glycoprotein, or glycolipid enriched in the Golgi body; (ii) detecting the first fluorophore; (iii) cleaving the disulfide bond in the first probe by addition of a reducing agent, thereby removing the first fluorophore from the sample; (iv) contacting the sample with a second probe comprising (a) a binding agent capable of binding to the third analyte, and (b) a second fluorophore, optionally wherein the binding agent and the second fluorophore are conjugated by a second cleavable linker comprising a disulfide bond; and (v) detecting the second fluorophore which, if present, is indicative of the presence or absence, amount, location, morphology and/or spatial patterning of the third analyte in the sample.

In some embodiments, the reducing agent is selected from the group consisting of DTT, TCEP, and BME. In some embodiments, the binding agent is a protein. In some embodiments, the binding agent is a lectin. In some embodiments, the binding agent is ConA. In some embodiments, the binding agent is a small molecule. In some embodiments, the binding agent is not a nucleic acid. In some embodiments, the first and second fluorophores produce the same or a similar detectable signal. In some embodiments, the first and second fluorophore are independently selected from the group consisting of an Alexa Fluor fluorophore, an ATTO dye, a cyanine dye, fluorescein, a fluorescent protein (e.g., GFP), a rhodamine dye, and a quenching agent (e.g., a Black Hole Quencher or an Iowa Black Quencher). In some embodiments, the first and second fluorophore are independently selected from the group consisting of Alexa Fluor 488, Alexa Fluor 555, Alexa Fluor 568, Alexa Fluor 594, Alexa Fluor 633, Alexa Fluor 647, and Alexa Fluor 750. In some embodiments, the first and second detectable fluorophores have substantially overlapping excitation and/or emission spectra. In some embodiments, the method further comprises: (vi) cleaving the second cleavable linker in the probe in contact with the sample, thereby removing the second fluorophore from the sample. In some embodiments, the second reducing agent is selected from the group consisting of DTT, TCEP, and BME.

Also disclosed herein, in certain embodiments, is a probe comprising a binding agent capable of binding to an analyte of interest, and a detectable label, wherein the binding agent and the detectable label are conjugated by a cleavable linker comprising a disulfide bond. In some embodiments, the binding agent is a protein. In some embodiments, the binding agent is a lectin. In some embodiments, the binding agent is Concanavalin A (ConA) or wheat germ agglutinin (WGA). In some embodiments, the binding agent is a small molecule.

In some embodiments, the detectable label is a fluorophore. In some embodiments, the fluorophore is selected from an Alexa fluorophore, an ATTO dye, a cyanine dye, fluorescein, a fluorescent protein (e.g., GFP), a rhodamine dye, and a quenching agent (e.g., a Black Hole Quencher or an Iowa Black Quencher). In some embodiments, the fluorophore is selected from Alexa Fluor 488, Alexa Fluor 555, Alexa Fluor 568, Alexa Fluor 595, Alexa Fluor 633, Alexa Fluor 647, and Alexa Fluor 750.

Also disclosed herein, in certain embodiments, is a probe comprising ConA, and a fluorophore, wherein ConA and the fluorophore are conjugated by a cleavable linker comprising a disulfide bond.

These and other aspects and features of the invention are described in the following detailed description and claims.

### DESCRIPTION OF THE DRAWINGS

The invention can be more completely understood with reference to the following drawings. It is noted that wherever practicable similar or like reference numbers may be used in the figures and may indicate similar or like functionality.

FIG. 1 depicts a general method for multiplexed imaging to re-image the same sample multiple times in the same fluorescent channel, according to some embodiments.

FIGS. 2A-2G depict various methods of removing conjugated fluorescent dyes from probes using a cleavable linker. FIG. 2A depicts removal of a single fluorescent dye covalently conjugated to a probe via a chemically cleavable linker. FIG. 2B depicts removal of multiple fluorescent dyes covalently conjugated to a probe via a chemically cleavable linker. FIG. 2C depicts removal of a single fluorescent dye covalently conjugated to a probe via a photocleavable linker. FIG. 2D depicts removal of a single fluorescent dye covalently conjugated to a probe via a linker that contains an oligonucleotide, such that the fluorophore can be removed using a specific chemical, light, or nuclease. FIG. 2E depicts removal of a single fluorescent dye covalently conjugated to a probe via an oligonucleotide bridge, which hybridizes to another nucleic acid connected directly or indirectly to the fluorophore, such that the fluorophore can be removed through chemical cleavage, photo cleavage, or nuclease cleavage. FIG. 2F depicts removal of a single fluorescent dye covalently conjugated to a probe via an oligonucleotide bridge, which hybridizes to another nucleic acid connected directly or indirectly to the fluorophore. Cleavage is performed using a nuclease or conditions that remove the fluorophore from the bridging oligonucleotide. FIG. 2G depicts removal of a single fluorescent dye covalently conjugated to a probe via a protein or small molecule-based bridge. Here, e.g., a biotin is covalently linked to the probe, and a cleavable fluorophore can effectively be conjugated.

FIGS. 3A-3F depict conjugation of protein-based probes (e.g., lectins such as Wheat Germ Agglutinin (WGA) and Concanavilin A (ConA)) to fluorescent dyes using chemically cleavable linkers to allow detection and subsequent removal of the dye signal. FIG. 3A depicts a general chemical structure of a probe covalently linked to a dye via a chemical linker containing a disulfide bond. FIG. 3B depicts fluorescence microscopy images of cells probed with WGA-Alexa Fluor 555 and ConA-Alexa Fluor 647, with dyes linked to each probe with a chemical linker containing a disulfide bond. Panels on the left show images prior to cleavage of the linker; panels on the right show the same cell samples following cleavage of the linkers. FIG. 3C depicts a histogram of the intensity per cell of Hoechst in images from FIG. 3B. FIG. 3D depicts a histogram of the intensity per cell of MitoTracker Green in images from FIG. 3B. FIG. 3E depicts a histogram of the intensity per cell of WGA-S-S-Alexa555 in images from FIG. 3B. FIG. 3F depicts a histogram of the intensity per cell of ConA-S-S-Alexa647 in images from FIG. 3B.

FIGS. 4A-4C depict fluorescence microscopy images of cells stained with probes conjugated to fluorescent dyes with cleavable linkers that were subsequently cleaved, and cells were stained with an additional fluorescent probe. FIG. 4A shows Hoescht and ConA-S-S-Alexa Fluor 647. The Alexa Fluor 647 was subsequently chemically cleaved (using the reducing agent TCEP in this case). FIG. 4B shows the same fields of view following cleavage of the linker and subsequent rest-staining of a gene-specific mRNA conjugated to Alexa Fluor 647. FIG. 4C shows merged images of the Alexa Fluor 647 channel detected in FIGS. 4A and 4B.

FIGS. 5A-5B depict fluorescent microscopy images of a mouse FFPE lung tumor sample from a human-patient derived xenograft, stained with probes conjugated to fluorescent dyes via cleavable disulfide linkers. Cells were simultaneously stained with WGA conjugated to Alexa Fluor 555 via a disulfide linker (“WGA-S-S-Alexa555”), with ConA conjugated to Alexa Fluor 647 via a disulfide linker (“ConA-S-S-Alexa647”), and with Hoechst to visualize DNA. Tissue sections were imaged before (FIG. 5A) and after (FIG. 5B) treatment with the reducing agent TCEP. All scale bars in the bottom right corners represent 100 microns.

### DETAILED DESCRIPTION

The present disclosure relates, in general, to methods and compositions for detecting analytes in a sample. Provided herein, for example, are methods and compositions for detecting multiple different analytes in a sample via imaging.

In one aspect, the disclosure provides a method for detecting the presence or absence, amount, location, morphology and/or spatial patterning of an analyte in a sample. In some embodiments, the contemplated method may comprise contacting the sample with a probe comprising a binding agent capable of binding to an analyte and a detectable label, where the binding agent and analyte are conjugated by a cleavable linker (e.g., a cleavable linker comprising a disulfide bond).

Various features and aspects of the disclosure are discussed in more detail below.

### I. Methods

Disclosed herein, in part, are methods for the detection of multiple different analytes within a biological sample via imaging.

In one aspect, the present disclosure provides a method for detecting the presence or absence, amount, location, morphology and/or spatial patterning of at least a first and a second analyte in a sample, the method comprising:
(i) contacting the sample with a first probe comprising (a) a first binding agent capable of binding to the first analyte, and (b) a first detectable label, wherein the first binding agent and the first detectable label are conjugated by a first cleavable linker (for example, comprising a disulfide bond, an ultraviolet (UV) light-sensitive moiety, or an enzymatically cleavable moiety such as an oligonucleotide or peptide) under conditions to permit the first probe to bind the first analyte if the first analyte is present in the sample;
(ii) detecting the first detectable label which, if present, is indicative of the presence or absence, amount, location, morphology and/or spatial patterning of the first analyte in the sample;
(iii) cleaving the first cleavable linker in the first probe, thereby removing the first detectable label from the sample;
(iv) contacting the sample with a second probe comprising (a) a second binding agent capable of binding to the second analyte, and (b) a second detectable label, optionally wherein the second binding agent and the second detectable label are conjugated by a second cleavable linker, under conditions to permit the second probe to bind the second analyte if the second analyte is present in the sample; and
(v) detecting the second detectable label which, if present, is indicative of the presence or absence, amount, location, morphology and/or spatial patterning of the second analyte in the sample.

The contemplated methods may detect the presence or absence, amount, location, morphology and/or spatial patterning of at least 50 analytes, at least 40 analytes, at least 30 analytes, at least 20 analytes, at least 15 analytes, at least 10 analytes, at least 9 analytes, at least 8 analytes, at least 7 analytes, at least 6 analytes, at least 5 analytes, at least 4 analytes, at least 3 analytes, or at least 2 analytes.

In some embodiments, the first and the second detectable labels are the same.

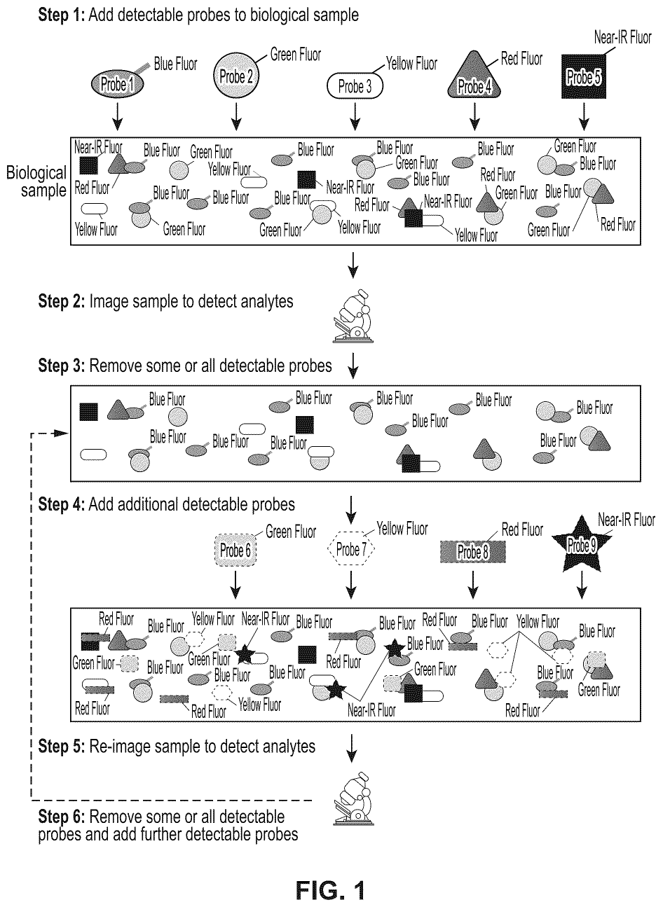

Reference is made to FIG. 1, which depicts an exemplary method for detecting multiple analytes within a sample (e.g., a biological sample). In Step (i) (corresponding to step 1 in FIG. 1), a sample is contacted using a detectable probe (e.g., a probe as described herein) to analyze an analyte, wherein the probe comprises a binding agent linked directly or indirectly to a detectable label (e.g., a fluorophore) by a cleavable linker. In some embodiments, the cleavable linker is a chemically cleavable linker, a photo-cleavable linker, or an enzymatically cleavable linker. In some embodiments, the cleavable linker comprises a disulfide bond.

In certain embodiments, step (i) comprises contacting the sample with more than one detectable probes. For example, in certain embodiments, the sample is contacted with 1 or more detectable probes, 2 or more detectable probes, 3 or more detectable probes, 4 or more detectable probes, 5 or more detectable probes, 6 or more detectable probes, 7 or more detectable probes, 8 or more detectable probes, 9 or more detectable probes, 10 or more detectable probes, 11 or more detectable probes, 12 or more detectable probes, 13 or more detectable probes, 14 or more detectable probes, or 15 or more detectable probes. In some embodiments, the sample is contacted with 1 or more detectable probes. In some embodiments, the sample is contacted with 2 or more detectable probes. In some embodiments, the sample is contacted with 3 or more detectable probes. In some embodiments, the sample is contacted with 4 or more detectable probes. In some embodiments, the sample is contacted with 5 or more detectable probes. In some embodiments, the sample is contacted with 6 or more detectable probes. In some embodiments, the sample is contacted with 7 or more detectable probes. In some embodiments, the sample is contacted with 8 or more detectable probes. In some embodiments, the sample is contacted with 9 or more detectable probes. In some embodiments, the sample is contacted with 10 or more detectable probes. In some embodiments, the sample is contacted with 11 or more detectable probes. In some embodiments, the sample is contacted with 12 or more detectable probes. In some embodiments, the sample is contacted with 13 or more detectable probes. In some embodiments, the sample is contacted with 14 or more detectable probes. In some embodiments, the sample is contacted with 15 or more detectable probes. In some embodiments, each detectable probe used to contact the sample during step (i) comprises the same cleavable linker (e.g., the same chemically cleavable linker, e.g., a linker comprising a disulfide bond).

In certain embodiments, the sample (e.g., biological sample) is selected from a tissue sample, a liquid sample, and a cell sample. In some embodiments, the biological sample is a tissue sample. In some embodiments, the biological sample is a liquid sample. In some embodiments, the sample is a cell sample. In some embodiments, the sample is a two-dimensional cell culture sample. In some embodiments, the sample is a three-dimensional cell culture sample. In some embodiments, the sample is a suspension cell culture sample. In some embodiments, the sample is an organoid sample. In some embodiments, the sample is a heterogeneous cell culture sample. In some embodiments, the sample is a mammalian cell and/or tissue sample. In some embodiments, the sample is a mouse cell and/or tissue sample. In some embodiments, the sample is a human cell and/or tissue sample. In some embodiments, the sample is a patient-derived cell or tissue sample. In some embodiments, the sample is obtained or derived from a biopsy. In some embodiments, the sample is a fresh sample. In some embodiments, the sample is a frozen sample. In some embodiments, the sample is a fixed sample, e.g., a chemically fixed sample. In some embodiments, the sample is a formalin-fixed, paraffin-embedded (FFPE) tissue sample. In some embodiments, the sample is a sectioned. In some embodiments, the sample comprises a healthy cell and/or a healthy tissue. In some embodiments, the sample comprises a malignant cell. In some embodiments, the sample comprises a cancer cell. In some embodiments, the sample comprises a tumor cell or tissue. In some embodiments, the sample comprises an infected cell. In some embodiments, the sample comprises a pathogen, e.g., a pathogen selected from a bacterial cell, a fungal cell, a virus, or a virally infected cell. In some embodiments, the sample comprises a genetically modified cell, e.g., a cell modified to express an ectopic nucleic acid and/or protein. In some embodiments, the sample is mounted on, adhered to, and/or immobilized on a surface, e.g. a microscopy slide.

In Step (ii) (corresponding to Step 2 in FIG. 1), the sample is analyzed for the presence of the detectable labels. For example, in some embodiments, the sample is imaged using an imaging device, e.g., a fluorescence microscope. Capturing images with the imaging device allows determination of the presence or absence, amount, location, morphology and/or spatial patterning of analytes bound by the detectable probes. The presence or absence of an analyte can be measured by the detection or non-detection of a signal, e.g., fluorescence, from a detectable probe for said analyte. The amount of an analyte can be measured, e.g., by quantifying a detected signal from a detectable probe specific for an analyte and comparing the quantified signal to an appropriate control or benchmark. Location of an analyte can be measured by detecting spatial localization of a signal from a probe specific for a first analyte with a signal from a probe for a known reference. For example, the location of an analyte within the nucleus may be measured by detection of a signal from a probe specific to the analyte overlapping with a signal from a probe specific for a nuclear component e.g., the Hoechst nucleic acid marker.

“Morphology” as used herein refers to the shape or structure of an analyte as measured by imaging. Morphology can refer to two-dimensional or three-dimensional shapes and structures. Morphology can also refer to intracellular or extracellular structures. For example, detection of the signal from a probe specific for the plasma membrane might indicate a bubbled morphology in cells undergoing apoptosis known as “blebbing.”

“Spatial patterning” as used herein refers to the overall distribution of an analyte within a sample as measured by detection of signal from probes specific for the analyte. Spatial patterning can include subcellular localization, e.g., localization relative to other probes or analytes, distribution relative to organelles, distribution relative to cellular polarity (e.g., apicobasal, planar, or migrational cell polarity), or distribution relative to extracellular features (e.g., the extracellular matrix or other cells). For example, a probe specific for endosomes may indicate punctate spatial patterning, whereas a probe for microtubules may indicate a filamentous spatial patterning.

In Step (iii) (corresponding to Step 3 in FIG. 1), all or a subset of the detectable labels are removed, enabling re-imaging using detectable labels with the same or similar properties as the removed detectable labels, e.g., fluorophores with substantially overlapping excitation/emissions spectra. In certain embodiments, the detectable labels are removed by cleavage of linkers conjugating the detectable label to the binding agent. In some embodiments, the linker is cleaved through chemical cleavage. In some embodiments, the linker is cleaved through photo-cleavage. In some embodiments, the linker is cleaved through enzymatic cleavage, e.g., by nuclease-mediated cleavage of an oligonucleotide linker. In some embodiments (for example, wherein the linker is a chemically cleavable linker, e.g., a linker comprising a disulfide bond), the linker is cleaved by contacting the sample with a reducing agent (e.g., dithiothreitol (DTT), tris(2-carboxyethyl) phosphine (TCEP), and/or β-mercaptoethanol (BME)). In some embodiments (for example, wherein the linker is a photo-cleavable linker), the linker is cleaved by exposing the sample to light (e.g., specific wavelengths, broad wavelengths, laser light, LED light, or ultraviolet (UV) light). In some embodiments (for example, wherein the linker is an enzymatically cleavable linker, e.g., an oligonucleotide), the linker is cleaved by contacting the sample with an appropriate enzyme (e.g., a sequence-specific nuclease, a sequence-independent nuclease, a restriction endonuclease, an exonuclease, an ssDNA nuclease, or an ssRNA nuclease).

In Step (iv) (corresponding to Step 4 in FIG. 1), the sample is re-contacted with a new detectable probe as described herein, e.g., a probe comprising a binding agent and a detectable label. In some embodiments, the analyte bound by the new probe is the same as an analyte bound by a probe applied in step (i). In some embodiments, the analyte bound by the new probe is different than the analyte(s) bound by the probe(s) applied in step (i).

In some embodiments, the binding agent of the new probe is different from the binding agent(s) of the one or more probe(s) applied in step (i). In some embodiments, the binding agent of the new probe is the same as the binding agent(s) of the one or more probe(s) applied in step (i). In some embodiments, the binding agent of the new probe is a protein, or the binding agent of a probe applied in step (i) is a protein. In some embodiments, the binding agent of the new probe and the binding agent of a probe applied in step (i) are both proteins. In some embodiments, the binding agent of the new probe is a lectin, or the binding agent of a probe applied in step (i) is a lectin. In some embodiments, the binding agent of the new probe and the binding agent of a probe applied in step (i) are both lectins. In some embodiments, the binding agent of the new probe or the binding agent of a probe applied in step (i) is independently selected from the group consisting of Concanavalin A (ConA), wheat germ agglutinin (WGA), Isolectin GS-IB4, Lectin GS-II, and PNA lectin. In some embodiments, the binding agent of the new probe and the binding agent of a probe applied in step (i) are each independently selected from the group consisting of Concanavalin A (ConA), wheat germ agglutinin (WGA), Isolectin GS-IB4, Lectin GS-II, and PNA lectin. In some embodiments, the binding agent of the new probe is ConA, or the binding agent of a probe applied in step (i) is ConA. In some embodiments, the binding agent of the new probe and the binding agent of a probe applied in step (i) are both ConA. In some embodiments, the binding agent of the new probe is WGA, or the binding agent of a probe applied in step (i) is WGA. In some embodiments, the binding agent of the new probe and the binding agent of a probe applied in step (i) are both WGA. In some embodiments, the binding agent of the new probe is a small molecule, or the binding agent of a probe applied in step (i) is a small molecule. In some embodiments, the binding agent of the new probe and the binding agent of a probe applied in step (i) are both small molecules. In some embodiments, the binding agent of the new probe is not a nucleic acid, or the binding agent of a probe applied in step (i) is not a nucleic acid. In some embodiments, neither the binding agent of the new probe nor the binding agent of a probe applied in step (i) are nucleic acids.

In some embodiments, the detectable label of the new probe is a fluorophore, e.g., a fluorophore selected from an Alexa Fluor fluorophore, an ATTO dye, a cyanine dye, fluorescein, a fluorescent protein (e.g., GFP), a rhodamine dye, and a quenching agent (e.g., a Black Hole Quencher or an Iowa Black Quencher). In some embodiments, the detectable label of the new probe is a fluorophore selected from Alexa Fluor 488, Alexa Fluor 555, Alexa Fluor 568, AlexaFluor 594, Alexa Fluor 633, Alexa Fluor 647, and Alexa Fluor 750. In some embodiments, the detectable label of the new probe is the same as or similar to the detectable label of a probe applied in step (i). In some embodiments wherein the detectable label of the new probe and the detectable label of a probe applied in step (i) are both fluorophores, the fluorophores have substantially overlapping excitation and/or emission spectra.

In some embodiments, the new detectable probe applied in step (iv) is a cleavable probe as described herein, e.g., a probe comprising a binding agent directly or indirectly linked to a detectable label by a cleavable linker. In some embodiments, the cleavable linker of the new probe is a chemically cleavable linker, a photo-cleavable linker, or an enzymatically cleavable linker. In some embodiments, the new probe comprises the same cleavable linker as the linker of a probe applied in step (i).

In certain embodiments, step (iv) comprises contacting the sample with more than one detectable probes. For example, in certain embodiments, the sample is contacted with 1 or more detectable probes, 2 or more detectable probes, 3 or more detectable probes, 4 or more detectable probes, 5 or more detectable probes, 6 or more detectable probes, 7 or more detectable probes, 8 or more detectable probes, 9 or more detectable probes, 10 or more detectable probes, 11 or more detectable probes, 12 or more detectable probes, 13 or more detectable probes, 14 or more detectable probes, or 15 or more detectable probes. In some embodiments, the sample is contacted with 1 or more detectable probes. In some embodiments, the sample is contacted with 2 or more detectable probes. In some embodiments, the sample is contacted with 3 or more detectable probes. In some embodiments, the sample is contacted with 4 or more detectable probes. In some embodiments, the sample is contacted with 5 or more detectable probes. In some embodiments, the sample is contacted with 6 or more detectable probes. In some embodiments, the sample is contacted with 7 or more detectable probes. In some embodiments, the sample is contacted with 8 or more detectable probes. In some embodiments, the sample is contacted with 9 or more detectable probes. In some embodiments, the sample is contacted with 10 or more detectable probes. In some embodiments, the sample is contacted with 11 or more detectable probes. In some embodiments, the sample is contacted with 12 or more detectable probes. In some embodiments, the sample is contacted with 13 or more detectable probes. In some embodiments, the sample is contacted with 14 or more detectable probes. In some embodiments, the sample is contacted with 15 or more detectable probes. In some embodiments, each detectable probe used to contact the sample during step (iv) comprises a cleavable linker. In some embodiments, each detectable probe used to contact the sample during step (iv) comprises the same cleavable linker (e.g., the same chemically cleavable linker, e.g., a linker comprising a disulfide bond).

In Step (v) (corresponding to Step 5 of FIG. 1), the sample is analyzed for the presence of the detectable label(s) of the new probe(s). For example, in some embodiments, the sample is imaged using an imaging device, e.g., a fluorescence microscope. In some embodiments, the sample is analyzed or imaged using the same method(s) as used in step (ii). As described in step (ii) hereinabove, the presence, absence, amount, location, morphology and/or spatial patterning of an analyte can be measured by detection or non-detection, e.g., fluorescence, from the new detectable probe for said analyte.

In certain embodiments, methods of the disclosure further comprise a Step (vi), wherein all or a subset of the detectable labels of the new detectable probes applied in step (iv) are removed. This subsequent removal enables further re-imaging using detectable labels with the same or similar properties as the removed detectable labels, e.g., using fluorophores with substantially overlapping excitation/emissions spectra. As described in step (iii) hereinabove, in certain embodiments, the detectable labels of the probes are removed by cleavage of linkers conjugating the detectable label to the binding agent. In some embodiments, the linker is cleaved through chemical cleavage. In some embodiments, the linker is cleaved through photo-cleavage. In some embodiments, the linker is cleaved through enzymatic cleavage, e.g., by nuclease-mediated cleavage of an oligonucleotide linker. In some embodiments (for example, wherein the linker is a chemically cleavable linker, e.g., a linker comprising a disulfide bond), the linker is cleaved by contacting the sample with a reducing agent (e.g., DTT, TCEP, and/or BME). In some embodiments (for example, wherein the linker is a photo-cleavable linker), the linker is cleaved by exposing the sample to light (e.g., specific wavelengths, broad wavelengths, laser light, or LED light). In some embodiments (for example, wherein the linker is an enzymatically cleavable linker, e.g., an oligonucleotide), the linker is cleaved by contacting the sample with an appropriate enzyme (e.g., a sequence-specific nuclease, a sequence-independent nuclease, a restriction endonuclease, an exonuclease, an ssDNA nuclease, or an ssRNA nuclease). In some embodiments, the detectable labels are removed in step (vi) using the same method as used in step (iii).

In certain embodiments, following removal of the detectable labels in step (vi), steps (iv) and (v) may be repeated using one or more further detectable probes (optionally, cleavable detectable probes) to enable imaging of additional probes to detect the same or different analytes. Steps (vi), (iv), and (v) can be iteratively repeated to enable imaging of additional probes to detect the same or different analytes. In some embodiments, the removal of detectable labels, re-probing, and re-imaging is iteratively repeated 1 or more times, 2 or more times, 3 or more times, 4 or more times, 5 or more times, 6 or more times, 7 or more times, 8 or more times, 9 or more times, 10 or more times, 15 or more times, 20 or more times, 25 or more times, 30 or more times, 35 or more times, 40 or more times, 45 or more times, or 50 or more times. In some embodiments, the removal of detectable labels, re-probing, and re-imaging is iteratively repeated 1 or more times. In some embodiments, the removal of detectable labels, re-probing, and re-imaging is iteratively repeated 2 or more times. In some embodiments, the removal of detectable labels, re-probing, and re-imaging is iteratively repeated 3 or more times. In some embodiments, the removal of detectable labels, re-probing, and re-imaging is iteratively repeated 4 or more times. In some embodiments, the removal of detectable labels, re-probing, and re-imaging is iteratively repeated 5 or more times. In some embodiments, the removal of detectable labels, re-probing, and re-imaging is iteratively repeated 6 or more times. In some embodiments, the removal of detectable labels, re-probing, and re-imaging is iteratively repeated 7 or more times. In some embodiments, the removal of detectable labels, re-probing, and re-imaging is iteratively repeated 8 or more times. In some embodiments, the removal of detectable labels, re-probing, and re-imaging is iteratively repeated 9 or more times. In some embodiments, the removal of detectable labels, re-probing, and re-imaging is iteratively repeated 10 or more times. In some embodiments, the removal of detectable labels, re-probing, and re-imaging is iteratively repeated 15 or more times. In some embodiments, the removal of detectable labels, re-probing, and re-imaging is iteratively repeated 20 or more times. In some embodiments, the removal of detectable labels, re-probing, and re-imaging is iteratively repeated 25 or more times. In some embodiments, the removal of detectable labels, re-probing, and re-imaging is iteratively repeated 30 or more times. In some embodiments, the removal of detectable labels, re-probing, and re-imaging is iteratively repeated 35 or more times. In some embodiments, the removal of detectable labels, re-probing, and re-imaging is iteratively repeated 40 or more times. In some embodiments, the removal of detectable labels, re-probing, and re-imaging is iteratively repeated 45 or more times. In some embodiments, the removal of detectable labels, re-probing, and re-imaging is iteratively repeated 50 or more times.

Also provided herein is a method for detecting the presence or absence, amount, location, morphology and/or spatial patterning of at least a first and a second analyte in a sample, wherein the first analyte is the endoplasmic reticulum (ER) and/or a protein, carbohydrate, glycoprotein, or glycolipid enriched in the ER, the method comprising:
(i) contacting the sample with a first probe comprising (a) ConA, and (b) a first fluorophore, wherein ConA and the first fluorophore are conjugated by a cleavable linker comprising a disulfide bond, under conditions to permit ConA to bind the ER and/or the protein, carbohydrate, glycoprotein, or glycolipid enriched in the ER;
(ii) detecting the first fluorophore;
(iii) cleaving the disulfide bond in the first probe by addition of a reducing agent, thereby removing the first fluorophore from the sample;
(iv) contacting the sample with a second probe comprising (a) a binding agent capable of binding to the second analyte, and (b) a second fluorophore, optionally wherein the binding agent and the second fluorophore are conjugated by a cleavable linker comprising a disulfide bond; and
(v) detecting the second fluorophore which, if present, is indicative of the presence, amount, location, morphology and/or spatial patterning of the second analyte in the sample.

In another aspect, provided herein is a method for detecting the presence or absence, amount, location, morphology and/or spatial patterning of at least a first, a second, and a third analyte in a sample, wherein the first analyte is a plasma membrane and/or a protein, carbohydrate, glycoprotein, or glycolipid enriched in the plasma membrane and the second analyte is a Golgi body and/or a protein, carbohydrate, glycoprotein, or glycolipid enriched in the Golgi body, the method comprising:
(i) contacting the sample with a first probe comprising (a) WGA, and (b) a first fluorophore, wherein WGA and the first fluorophore are conjugated by a cleavable linker comprising a disulfide bond, under conditions to permit WGA to bind the plasma membrane and/or the protein, carbohydrate, glycoprotein, or glycolipid enriched in the plasma membrane and the Golgi body and/or the protein, carbohydrate, glycoprotein, or glycolipid enriched in the Golgi body;
(ii) detecting the first fluorophore;
(iii) cleaving the disulfide bond in the first probe by addition of a reducing agent, thereby removing the first fluorophore from the sample;
(iv) contacting the sample with a second probe comprising (a) a binding agent capable of binding to the third analyte, and (b) a second fluorophore, optionally wherein the binding agent and the second fluorophore are conjugated by a cleavable linker comprising a disulfide bond; and
(v) detecting the second fluorophore which, if present, is indicative of the presence or absence, amount, location, morphology and/or spatial patterning of the third analyte in the sample.

### II. Analytes

The systems and methods described herein may be used to detect the presence, or to quantify the amount, of an analyte in a sample of interest, for example, a cell sample, a liquid sample, or a tissue sample.

Analytes may be detected and/or quantified in a variety of samples. In certain embodiments, the sample is derived from a subject. As used herein, the terms “subject” and “patient” refer to an organism that is the source of a sample that is interrogated by the methods described herein Such organisms preferably include, but are not limited to, mammals (e.g., murines, simians, equines, bovines, porcines, canines, felines, and the like), and more preferably includes humans.

The sample can be in any form that allows for measurement of the analyte. In other words, the sample must be sufficient for analyte extraction or processing to permit detection of the analyte, such as preparation of thin sections. Accordingly, the sample can be fresh, preserved through suitable cryogenic techniques, or preserved through non-cryogenic techniques.

In certain embodiments, the sample is a body fluid sample, such as a blood, serum, plasma, urine, saliva, cerebrospinal fluid, or interstitial fluid sample.

In certain embodiments, the sample is a tissue sample, such as a biopsy sample. A biopsy sample can be obtained by using conventional biopsy instruments and procedures. Endoscopic biopsy, excisional biopsy, incisional biopsy, fine needle biopsy, punch biopsy, shave biopsy and skin biopsy are examples of recognized medical procedures that can be used by one of skill in the art to obtain tissue samples. A standard process for handling clinical biopsy tissue specimens is to fix the tissue sample in formalin and then embed the sample in paraffin. Samples in this form are commonly known as formalin-fixed, paraffin-embedded (FFPE) tissue. Suitable techniques of tissue preparation for subsequent analysis are well-known to those of skill in the art.

In certain embodiments, the sample is a cell sample, or a cell supernatant sample.

Exemplary analytes include cells, organic compounds, antibodies, antigens, virus particles, pathogenic bacteria, metals, metal complexes, ions, spores, yeasts, molds, cellular metabolites, enzyme inhibitors, receptor ligands, nerve agents, peptides, proteins, fatty acids, steroids, hormones, narcotic agents, synthetic molecules, medications, enzymes, nucleic acid single-stranded or double-stranded polymers. Analytes include biological molecules, for example, a protein, peptide, carbohydrate, glycoprotein, glycopeptide, lipid, lipoprotein, nucleic acid, or nucleoprotein. In some embodiments, the analyte is selected from the group consisting of a cell, organelle, protein, peptide, carbohydrate, glycoprotein, glycopeptide, glycolipid, lipid, lipoprotein, nucleic acid, and nucleoprotein. In certain embodiments, the analyte is selected from the group consisting of an endoplasmic reticulum, a plasma membrane, a Golgi body, a microtubule, an actin filament, a sarcomere, a collagen fibril, a condensate, a protein condensate, a nucleic acid condensate, a protein-nucleic acid mixture condensate, a membraneless organelle, a P-body, a Cajal body, a stress granule, a nuclear speckle.

In certain embodiments, the analyte is an organelle. Examples of organelles include, but are not limited to, the nucleus, the nucleolus, the plasma membrane, the endoplasmic reticulum (ER), the smooth ER, the rough ER, the Golgi body, an endosome, an early endosome, a late endosome, a recycling endosome, an autophagosome, an autolysosome, a lysosome, a peroxisome, a ribosome, a condensate, a protein condensate, a nucleic acid condensate, a protein-nucleic acid mixture condensate, a membraneless organelle, a P-body, a Cajal body, a stress granule, a nuclear speckle, a mitochondrion, a chloroplast, a microtubule, an actin filament, an intermediate filament, a vesicle, a centriole, an exosome, a cellular junction, a cilium, or a flagellum.

In certain embodiments the analyte is a cytokine. Examples of cytokines include, but are not limited to, interferons (e.g., IFNα, IFNβ, and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In certain embodiments, the analyte is a carbohydrate, a glycoprotein, and/or a glycolipid. Examples of carbohydrates, glycoproteins, and glycolipids include, but are not limited to carbohydrates, glycoproteins, or glycolipids comprising lactose, D-mannose, D-glucose, D-fucose, L-fucose (e.g. alpha-L-fucose), D-galactose, blood group A oligosaccharides, blood group B oligosaccharides, saccharides comprising alpha-D-Gal(1→3)[alpha-Lfuc(1→2)]-beta-D-Gal(1→3/4-beta-D-GlcNAc, saccharides comprising alpha-sialyl [2→3]-lactose, alpha-D-mannosyl glycoconjugates, alpha-NeuNAc-[2→6]-Gal, alpha-NeuNAc-[2→6]-GalNAc, alpha-NeuNAc-[2→3]-Gal, N-acetyl-beta-D-glucosamine, terminal alpha-D-galactosyl residues, terminal beta-D-galactosyl residues, N-acetyllactosamine, terminal alpha-D-mannosyl residues, N-acetyl-beta-D-glucosamine, terminal N-acetyl-D-galactosamine, N-acetylneuraminic acid, and terminal alpha-D-galactosaminyl residues.

In certain embodiments the analyte is a hormone. Examples of hormones include, but are not limited to, epinephrine, melatonin, norepinephrine, triiodothyronine, thyroxine, dopamine, prostaglandins, leukotrienes, prostacyclin, thromboxane, amylin (or islet amyloid polypeptide), anti-Müllerian hormone (or Müllerian inhibiting factor or hormone), adiponectin, adrenocorticotropic hormone (or corticotropin), angiotensinogen and angiotensin, antidiuretic hormone (or vasopressin, arginine vasopressin), atrial-natriuretic peptide (or atriopeptin), brain natriureticc peptide, calcitonin, cholecystokinin, corticotropin-releasing hormone, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor (or somatomedin), leptin, lipotropin, luteinizing hormone, melanocyte stimulating hormone, motilin, orexin, osteocalcin, oxytocin, pancreatic polypeptide, parathyroid hormone, pituitary adenylate cyclase-activating peptide, prolactin, prolactin releasing hormone, relaxin, renin, secretin, somatostatin, thrombopoictin, thyroid-stimulating hormone (or thyrotropin), thyrotropin-releasing hormone, vasoactive intestinal peptide, guanylin, uroguanylin, testosterone, dehydroepiandrosterone, androstenedione, dihydrotestosterone, aldosterone, estradiol, estrone, estriol, cortisol, progesterone, calcitriol (1,25-dihydroxyvitamin D3), and calcidiol (25-hydroxyvitamin D3).

In certain embodiments the analyte is a cancer antigen. Examples of cancer antigens include, but are not limited to, adenosine A2a receptor (A2aR), A kinase anchor protein 4 (AKAP4), B melanoma antigen (BAGE), brother of the regulator of imprinted sites (BORIS), breakpoint cluster region Abelson tyrosine kinase (BCR/ABL), CA125, CAIX, CD19, CD20, CD22, CD30, CD33, CD52, CD73, CD137, carcinoembryonic antigen (CEA), CSI, cytotoxic T-lymphocyte-associated antigen 4 (CTLA-4), estrogen receptor binding site associated antigen 9 (EBAG9), epidermal growth factor (EGF), epidermal growth factor receptor (EGFR), EGF-like module receptor 2 (EMR2), epithelial cell adhesion molecule (EpCAM) (17-1A), FR-alpha, G antigen (GAGE), disialoganglioside GD2 (GD2), glycoprotein 100 (gp100), human epidermal growth factor receptor 2 (Her2), hepatocyte growth factor (HGF), human papillomavirus 16 (HPV-16), heat-shock protein 105 (HSP105), isocitrate dehydrogenase type 1 (IDH1), idiotype (NeuGcGM3), indoleamine-2,3-dioxygenase 1 (IDO1), IGF-1, IGF1R, IGG1K, killer cell immunoglobulin-like receptor (KIR), lymphocyte activation gene 3 (LAG-3), lymphocyte antigen 6 complex K (LY6K), Matrix-metalloproteinase-16 (MMP16), melanotransferrin (MF12), melanoma antigen 3 (MAGE-A3), melanoma antigen C2 (MAGE-C2), melanoma antigen D4 (MAGE-D4), melanoma antigen recognized by T-cells 1 (Melan-A/MART-1), N-methyl-N′-nitroso-guanidine human osteosarcoma transforming gene (MET), mucin 1 (MUC1), mucin 4 (MUC4), mucin 16 (MUC16), New York esophageal squamous cell carcinoma 1 (NY-ESO-1), prostatic acid phosphatase (PAP), programmed cell death receptor 1 (PD-1), programmed cell death receptor ligand 1 (PD-L1), phosphatidylserine, preferentially expressed antigen of melanoma (PRAME), prostate specific antigen (PSA), protein tyrosine kinase 7 (PTK7, also known as colon carcinoma kinase 4 (CCK4)), receptor tyrosine kinase orphan receptor 1 (ROR1), scatter factor receptor kinase, sialyl-Tn, sperm-associated antigen 9 (SPAG-9), synovial sarcoma X-chromosome breakpoint 1 (SSX1), survivin, telomerase, T-cell immunoglobulin domain and mucin domain-3 (TIM-3), vascular endothelial growth factor (VEGF) (e.g., VEGF-A), vascular endothelial growth factor Receptor 2 (VEGFR2), V-domain immunoglobulin-containing suppressor of T-cell activation (VISTA), Wilms' Tumor-1 (WT1), X chromosome antigen 1b (XAGE-1b), 5T4, Mesothelin, Glypican 3 (GPC3), Prostate Specific Membrane Antigen (PSMA), cMET, CD38, B Cell Maturation Antigen (BCMA), CD123, CLDN6, CLDN9, LRRC15, PRLR (Prolactin Receptor), RING finger protein 43 (RNF43), Uroplakin-1 B (UPK1 B), tumor necrosis factor superfamily member 9 (TNFSF9), tumor necrosis factor receptor superfamily member 21 (TNFSRF21), bone morphogenetic protein receptor type-1B (BMPR1B), Kringle domain-containing transmembrane protein 2 (KREMEN2), Delta-like protein 3 (DLL3), Siglec7 and Siglec9. Additional exemplary cancer antigens include those found on cancer stem cells, e.g., SSEA3, SSEA4, TRA-1-60, TRA-1-81, SSEA1, CD133 (AC133), CD90 (Thy-1), CD326 (EpCAM), Cripto-1 (TDGF1), PODXL-1 (Podocalyxin-like protein 1), ABCG2, CD24, CD49f (Integrin a6), Notch2, CD146 (MCAM), CD10 (Neprilysin), CD117 (c-KIT), CD26 (DPP-4), CXCR4, CD34, CD271, CD13 (Alanine aminopeptidase), CD56 (NCAM), CD105 (Endoglin), LGR5, CD114 (CSF3R), CD54 (ICAM-1), CXCR1, 2, TIM-3 (HAVCR2), CD55 (DAF), DLL4 (Delta-like ligand 4), CD20 (MS4A1), and CD96.

### III. Probes

### A. Binding Agents

In another aspect, the present disclosure provides a probe for detecting analytes. Probes useful in the practice of the disclosure include a binding agent. The term “binding agent” as used herein refers to an agent that binds preferentially or specifically to an analyte of interest. The terms “bind preferentially,” or “binds specifically” as used in connection with a binding agent refers to an agent that binds and/or associates (i) more stably, (ii) more rapidly, (iii) with stronger affinity, (iv) with greater duration, or (v) or a combination of any two or more of (i)-(iv), with a particular target analyte it does with a molecule other than the target analyte. For example, a binding agent that specifically or preferentially binds a target analyte is a binding domain that binds a target analyte, e.g., with stronger affinity, avidity, more readily, and/or with greater duration than it binds a different analyte. The binding agent have affinity for the analyte of about 100 nM, 50 nM, 20 nM, 15 nM, 10 nM, 9 nM, 8 nM, 7 nM, 6 nM, 5 nM, 4 nM, 3 nM, 2 nM, 1 nM, 0.5 nM, 0.1 nM, or 0.01 nM, or stronger, as determined by surface plasmon resonance. For example, the binding agent may have an affinity for the analyte within the range from about 0.01 nM to about 100 nM, from about 0.1 nM to about 100 nM, or from about 1 nM to about 100 nM. It is understood that a binding agent that binds preferentially to a first target analyte may or may not preferentially bind to a second target analyte. As such, “preferential binding” does not necessarily require (although it can include) exclusive binding.

Exemplary binding agents include proteins, for example, lectins (for example, that bind carbohydrates), enzymes (for example, that bind substrates and inhibitors), antibodies (for example, that bind antigens), antigens (for example, that bind target antibodies), receptors (for example, that bind ligands), ligands (for example, that bind receptors), nucleic acid single-strand polymers (for example, that bind nucleic acid molecules to form, for example, DNA-DNA, RNA-RNA, or DNA-RNA double strands), nucleic acid aptamers (for example, that bind to targets) and synthetic molecules that bind with target analytes. Natural, synthetic, semi-synthetic, and genetically-altered macromolecules may be employed as binding agents. In certain embodiments, the binding agent is a biological binding agent, for example, an antibody, an aptamer, a receptor, an enzyme, or a nucleic acid.

In certain embodiments, the binding agent is a lectin. Examples of lectins include, but are not limited to Concanavalin A (ConA), wheat germ agglutinin (WGA), Isolectin GS-IB4, Lectin GS-II, and PNA lectin. In some embodiments, the binding agent is ConA. In some embodiments, the binding agent is WGA. In some embodiments, the binding agent is Isolectin GS-IB4. In some embodiments, the binding agent is Lectin GS-II. In some embodiments, the binding agent is PNA.

As used herein, unless otherwise indicated, the term “antibody” is understood to mean an intact antibody (e.g., an intact monoclonal antibody) or antigen-binding fragment of an antibody (e.g., an antigen-binding fragment of a monoclonal antibody), including an intact antibody or antigen-binding fragment that has been modified, engineered, or chemically conjugated. Examples of antibodies that have been modified or engineered include chimeric antibodies, humanized antibodies, and multi-specific antibodies (e.g., bispecific antibodies). Examples of antigen-binding fragments include Fab, Fab′, (Fab′)₂, Fv, single chain antibodies (e.g., scFv), minibodies, and diabodies.

In certain embodiments, an antibody binds to its target with a K_{D} of about 300 pM, 250 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, or 10 pM, or lower.

An antibody may have a human IgG1, IgG2, IgG3, IgG4, or IgE isotype.

In certain embodiments, the antibody is selected from, or the antibody is derived from antibody selected from, adecatumumab, ascrinvacumab, cixutumumab, conatumumab, daratumumab, drozitumab, duligotumab, durvalumab, dusigitumab, enfortumab, cnoticumab, cpratuxumab, figitumumab, ganitumab, glembatumumab, intetumumab, ipilimumab, iratumumab, icrucumab, lexatumumab, lucatumumab, mapatumumab, narnatumab, necitumumab, nesvacumab, ofatumumab, olaratumab, panitumumab, patritumab, pritumumab, radretumab, ramucirumab, rilotumumab, robatumumab, seribantumab, tarextumab, teprotumumab, tovetumab, vantictumab, vesencumab, votumumab, zalutumumab, flanvotumab, altumomab, anatumomab, arcitumomab, bectumomab, blinatumomab, detumomab, ibritumomab, minretumomab, mitumomab, moxetumomab, naptumomab, nofctumomab, pemtumomab, pintumomab, racotumomab, satumomab, solitomab, taplitumomab, tenatumomab, tositumomab, tremelimumab, abagovomab, atczolizumab, durvalumab, avelumab, igovomab, oregovomab, capromab, edrecolomab, nacolomab, amatuximab, bavituximab, brentuximab, cetuximab, derlotuximab, dinutuximab, ensituximab, futuximab, girentuximab, indatuximab, isatuximab, margetuximab, rituximab, siltuximab, ublituximab, ccromeximab, abituzumab, alemtuzumab, bevacizumab, bivatuzumab, brontictuzumab, cantuzumab, cantuzumab, citatuzumab, clivatuzumab, dacetuzumab, demcizumab, dalotuzumab, denintuzumab, clotuzumab, emactuzumab, emibetuzumab, enoblituzumab, ctaracizumab, farletuzumab, ficlatuzumab, gemtuzumab, imgatuzumab, inotuzumab, labetuzumab, lifastuzumab, lintuzumab, lirilumab, lorvotuzumab, lumretuzumab, matuzumab, milatuzumab, moxctumomab, nimotuzumab, obinutuzumab, ocaratuzumab, otlertuzumab, onartuzumab, oportuzumab, parsatuzumab, pertuzumab, pidilizumab, pinatuzumab, polatuzumab, sibrotuzumab, simtuzumab, tacatuzumab, tigatuzumab, trastuzumab, tucotuzumab, urelumab, vandortuzumab, vanucizumab, veltuzumab, vorsetuzumab, sofituzumab, catumaxomab, ertumaxomab, depatuxizumab, ontuxizumab, blontuvetmab, tamtuvetmab, nivolumab, pembrolizumab, cpratuzumab, MEDI9447, urclumab, utomilumab, hu3F8, hu14.18-IL-2, 3F8/OKT3BsAb, lirilumab, BMS-986016 pidilizumab, AMP-224, AMP-514, BMS-936559, atezolizumab, and avelumab.

Where the binding agent comprises, or is derived from, a protein (e.g., an antibody), and the binding agent comprises a protein sequence comprising an unnatural amino acid (UAA), and an oligonucleotide is conjugated to the binding agent via the UAA, it is understood that the UAA may be incorporated (and the oligonucleotide may therefore be conjugated) to any appropriate location within the protein sequence.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) Nature Genetics 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PNAS 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

Typically, where the binding agent is an antibody or a lectin, between each assay step, the bound analyte is washed, for example, with a mild detergent solution. Protocols may also include one or more blocking steps, which involve use of a non-specifically-binding protein such as bovine serum albumin to block unwanted non-specific binding of protein reagents.

Methods for producing antibodies are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs encoding the desired antibodies. Production of defined gene constructs is within routine skill in the art.

Nucleic acids encoding desired antibodies can be incorporated (e.g., ligated) into suitable expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In some embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

Exemplary nucleic acid based binding agents include aptamers and spiegelmers. Aptamers are nucleic acid-based sequences that have strong binding activity for a specific target molecule. Spiegelmers are similar to aptamers with regard to binding affinities and functionality but have a structure that prevents enzymatic degradation, which is achieved by using nuclease resistant L-oligonucleotides rather than naturally occurring, nuclease sensitive D-oligonucleotides.

Aptamers are specific nucleic acid sequences that bind to target molecules with high affinity and specificity and are identified by a method commonly known as Selective Evolution of Ligands by Evolution (SELEX), as described, for example, in U.S. Pat. Nos. 5,475,096 and 5,270,163. Each SELEX-identified nucleic acid ligand is a specific ligand of a given target compound or molecule. The SELEX process is based on the observation that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

The SELEX method applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand enriched mixture of nucleic acids, then reiterating the steps of binding, partitioning, dissociating and amplifying through as many cycles as desired to yield highly specific high affinity nucleic acid ligands to the target molecule. Thus, this method allows for the screening of large random pools of nucleic acid molecules for a particular functionality, such as binding to a given target molecule.

The SELEX method also encompasses the identification of high-affinity nucleic acid ligands containing modified nucleotides conferring improved characteristics on the ligand, such as improved in vivo stability and protease resistance. Examples of such modifications include chemical substitutions at the ribose and/or phosphate and/or base positions. SELEX process-identified nucleic acid ligands containing modified nucleotides are described in U.S. Pat. Nos. 5,660,985 and 5,580,737, which include highly specific nucleic acid ligands containing one or more nucleotides modified at the 2′ position with, for example, a 2′-amino, 2′-fluoro, and/or 2′-O-methyl moiety.

In certain embodiments, it is contemplated that spiegelmers, mirror image aptamers composed of L-ribose or L-2′deoxyribose units (see, U.S. Pat. Nos. 8,841,431, 8,691,784, 8,367,629, 8,193,159 and 8,314,223) can be used in the practice of the disclosure. The chiral inversion in spiegelmers results in an improved plasma stability compared with natural D-oligonucleotide aptamers. L-nucleic acids are enantiomers of naturally occurring D-nucleic acids that are not very stable in aqueous solutions and in biological systems or samples due to the widespread presence of nucleases. Naturally occurring nucleases, particularly nucleases from animal cells are not capable of degrading L-nucleic acids. Because of this, the biological half-life of the L-nucleic acid is significantly increased in such a system, including the animal and human body. Due to the lacking degradability of L-nucleic acids, no nuclease degradation products are generated and thus no side effects arising therefrom observed.

Using in vitro selection, an oligonucleotide that binds to the synthetic enantiomer of a target molecule, e.g., a D-peptide, can be selected. The resulting aptamer is then resynthesized in the L-configuration to create a spiegelmer (from the German “spiegel” for mirror) that binds the physiological target with the same affinity and specificity as the original aptamer to the mirror-image target. This approach has been used to synthesize spiegelmers that bind, for example, hepcidin (see, U.S. Pat. No. 8,841,431), MCP-1 (see, U.S. Pat. Nos. 8,691,784, 8,367,629 and 8,193,159) and SDF-1 (see, U.S. Pat. No. 8,314,223).

In certain embodiments, the contemplated binding agents may not be a nucleic acid.

In certain embodiments, the binding agent is a small molecule, e.g., phalloidin.

### B. Detectable Labels

Probes useful in the practice of the disclosure comprise a detectable label, for example, a fluorescent dye comprising a fluorophore.

In some embodiments, probes of the present disclosure comprise a fluorescent dye.

Fluorescent dyes are widely used in biological research and medical diagnostics. In particular, a diversity of fluorophores with a distinguishable color range has made it more practical to perform multiplexed assays capable of detecting multiple biological targets at the same time. The ability to visualize multiple targets in parallel is often required for delineating the spatial and temporal relationships amongst different biological targets in vitro and in vivo.

In some embodiments, the fluorescent dye is an Alexa Fluor. Examples of Alexa Fluors

include, but are not limited to Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750, and Alexa Fluor 790.

In some embodiments, the fluorescent dye is a rhodamine dye. Examples of rhodamine dyes include, but are not limited to rhodamine, rhodamine 6G, rhodamine 123, rhodamine B, sulforhodamine 101, and sulforhodamine B.

In some embodiments, the fluorescent dye is a DyLight Fluor. Examples of DyLight Fluors include, but are not limited to DyLight 350, DyLight 405, DyLight 488, DyLight 550, DyLight 594, DyLight 633, DyLight 650, DyLight 680, DyLight 755, and DyLight 800.

In some embodiments, the fluorescent dye is a cyanine dye. Examples of cyanine dyes include, but are not limited to cyanine 2 (Cy2), cyanine 3 (Cy3), cyanine 3B (Cy3B), cyanine 3.5 (Cy3.5), cyanine 5 (Cy5), cyanine 5.5 (Cy5.5), cyanine 7 (Cy7), and cyanine 7.5 (Cy7.5).

In some embodiments, the fluorescent dye is an ATTO dye. Examples of ATTO dyes include, but are not limited to ATTO 390, ATTO 425, ATTO 465, ATTO 488, ATTO 495, ATTO 514, ATTO 520, ATTO 532, ATTO Rho6G, ATTO 540Q, ATTO 550, ATTO 565, ATTO Rho3B, ATTO Rho11, ATTO Rho12, ATTO Thio12, ATTO 580Q, ATTO Rho101, ATTO 590, ATTO Rho13, ATTO 594, ATTO 610, ATTO 612Q, ATTO 620, ATTO Rho14, ATTO 633, ATTO 647 ATTO 647N, ATTO 655, ATTO Oxa12, ATTO 665, ATTO 680, ATTO 700, ATTO 725, ATTO 740, and ATTO MB2.

Other examples of fluorescent dyes include, but are not limited to Freedom Dyes, Janelia Fluor Dyes, green fluorescent protein (GFP), red fluorescent protein (RFP), yellow fluorescent protein (YFP), blue fluorescent protein (BFP), cyan fluorescent protein (CFP), DSRed, cGFP, mEmerald, mWasabi, Azami Green, mAzurite, mCerulean, mTurquoise, mTopaz, mVenus, mCitrine, mBanana, Kusabia Orange, mOrange, dTomato, mTangerine, mRuby, mApple, mStrawberry, mCherry, mRaspberry, mPlum, fluorescein, phycoerythrin (PE), and peridinin chlorophyll protein (PerCP).

Fluorescent dyes can be detected by any suitable method known to those of ordinary skill in the art, preferably by fluorescence microscopy. In some embodiments, the fluorescence microscopy is wide-field fluorescence microscopy. In some embodiments, the fluorescence microscopy is laser scanning confocal microscopy. In some embodiments, the fluorescence microscopy is spinning disc confocal microscopy. In some embodiments, the fluorescence microscopy is two-photon microscopy. Methods of fluorescence microscopy are summarized in Sanderson et al., Cold Spring Harb Protoc. 2014 October; 2014 (10)

In some embodiments, the probes of the disclosure may be labeled with a radioisotope (i.e., radiolabeled). Probes can be radiolabeled either directly by incorporating the label directly into a probe of the disclosure or indirectly by incorporating the label into a probe of the disclosure through a chelating agent, where the chelating agent has been incorporated into the compound). Furthermore, a label for a probe can be included as an additional substituent (group, moiety, position) to a compound of the disclosure or as an alternative substituent for any substituents that are present. Exemplary radioisotopes can include ³H, ¹¹C, ¹⁴C, ¹⁸F, ³²P, ³⁵S, ¹²³I, ¹²⁵I, ¹³¹I, ¹²⁴I, ¹⁹F, ⁷⁵Br, ¹³C, ¹³N, ¹⁵O, ⁷⁶Br, or ⁹⁹Tc. A radiolabel may appear at any substituent (group, moiety, position) on a compound or probe of the disclosure. In other embodiments, the first and second detectable labels produce the same or similar detectable signal. In certain embodiments, the first and second detectable labels are fluorophores that have substantially overlapping excitation and/or emission spectra.

In certain embodiments, a probe of the disclosure comprises more than one detectable label. For example, in some embodiments, the probe comprises at least one, at least two, at least three, at least four, or at least five detectable labels. In some embodiments, the probe comprises one detectable label. In some embodiments, the probe comprises two detectable labels. In some embodiments, the probe comprises three detectable labels. In some embodiments, the probe comprises four detectable labels. In some embodiments, the probe comprises five detectable labels. In some embodiments, the probe comprises more than five detectable labels. In some embodiments, each detectable label is directly or indirectly linked to the binding agent of the probe by a cleavable linker.

### C. Cleavable Linker

Detectable probes for applications as described herein are generally derived from joining a detectable label and an agent that is capable of binding to a given target, analyte, or binding partner. Current processes for joining dyes to such binding agents, e.g., antibodies, typically require purification of the antibody away from standard buffer components, long reaction times, and purification of the labeled antibody once the reaction is complete. Thus, current processes consume valuable time and resources, and there is a need for improvements in the labeling of binding agents.

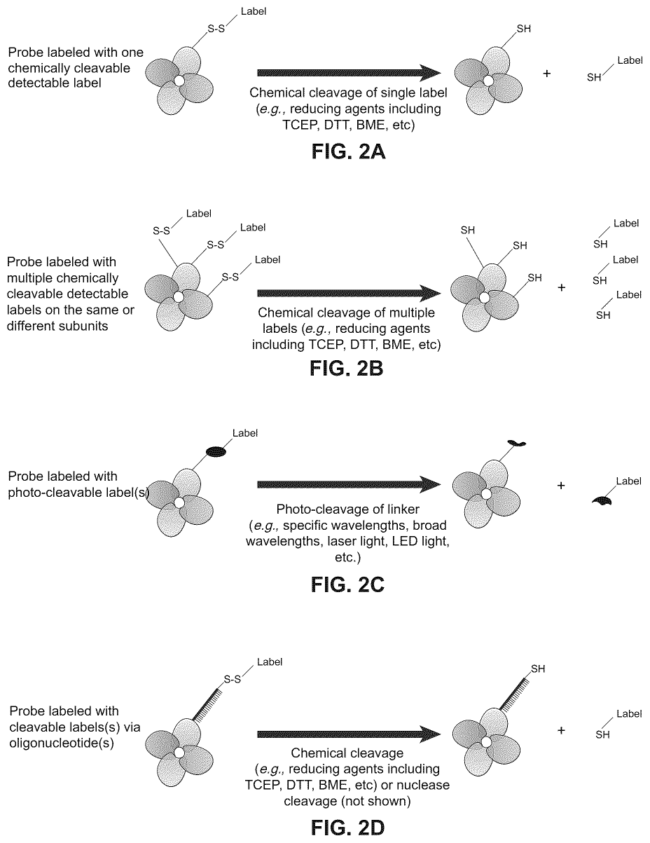

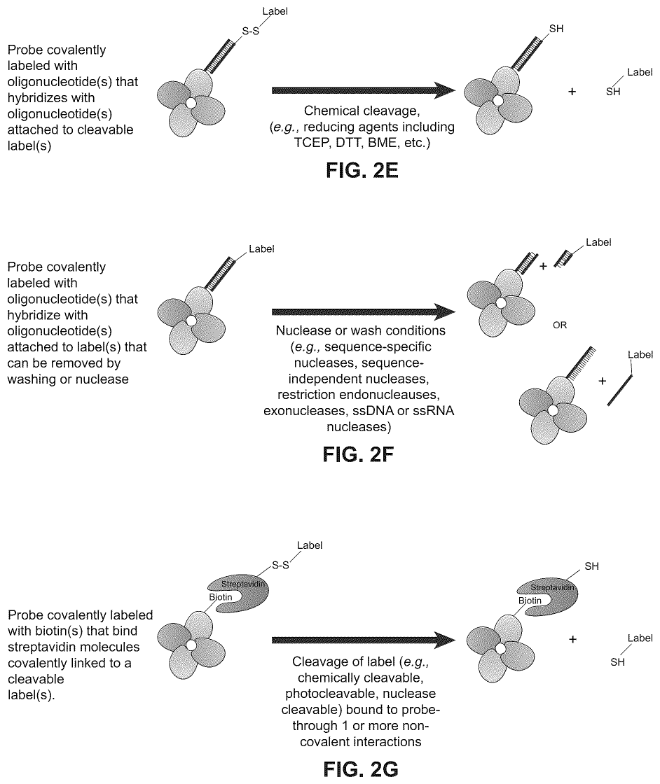

A contemplated probe contains a binding agent and a delectable label, which are conjugated (e.g., covalently coupled) by a cleavable linker. FIGS. 2A-2G outline several methods that can be used to couple cleavable fluorophores to probes. The contemplated linkers can be, for example, chemically cleavable, photo-cleavable, nuclease cleavable, or removable using specific wash conditions.

In some embodiments, the probe comprises a chemically cleavable linker. For example, a chemically cleavable linker can comprise a disulfide bond, which can be cleaved by adding a reducing agent. In some embodiments, the reducing agent is dithiothreitol (DTT), tris(2-carboxyethyl) phosphine (TCEP), or β-mercaptoethanol (BME). In some embodiments, the reducing agent is DTT. In some embodiments, the reducing agent is TCEP. In some embodiments, the reducing agent is BME.

In some embodiments, the probe comprises a photo-cleavable linker. A photo-cleavable linker can be cleaved by light exposure. In some embodiments, the linker can be cleaved by exposure to specific wavelengths of light, broad wavelengths of light, laser light, LED light, or ultraviolet (UV) light. In some embodiments, the linker can be cleaved by UV light.

In some embodiments, the probe comprises an enzymatically cleavable linker. For example, enzymatically cleavable linkers can comprise nucleic acids that can be cleaved by nucleases (e.g., restriction enzymes, sequence-specific nucleases, sequence-independent nucleases, exonucleases, ssDNA nucleases, or ssRNA nucleases) or peptides that can be cleaved by sequence-specific proteases.

Other modifications can also be made, including using one or more cleavable fluorophores per probe molecule to adjust the brightness per probe, coupling the cleavable fluorophore through covalent or non-covalent linkers. For example, linkers can have a biotin-streptavidin linkage between the probe and the cleavable fluorophore or have the cleavable fluorophore covalently linked to an oligonucleotide that base pairs to an oligonucleotide that is covalently linked to the probe.

In some embodiments, multiple probes with different types of linkers can be included in a single experiment. This can allow removal of specific probes without affecting other probes. This is distinct from commonly used nucleic acid-based multiplexing wherein all cleavable FISH-based constructs have a similar structure.

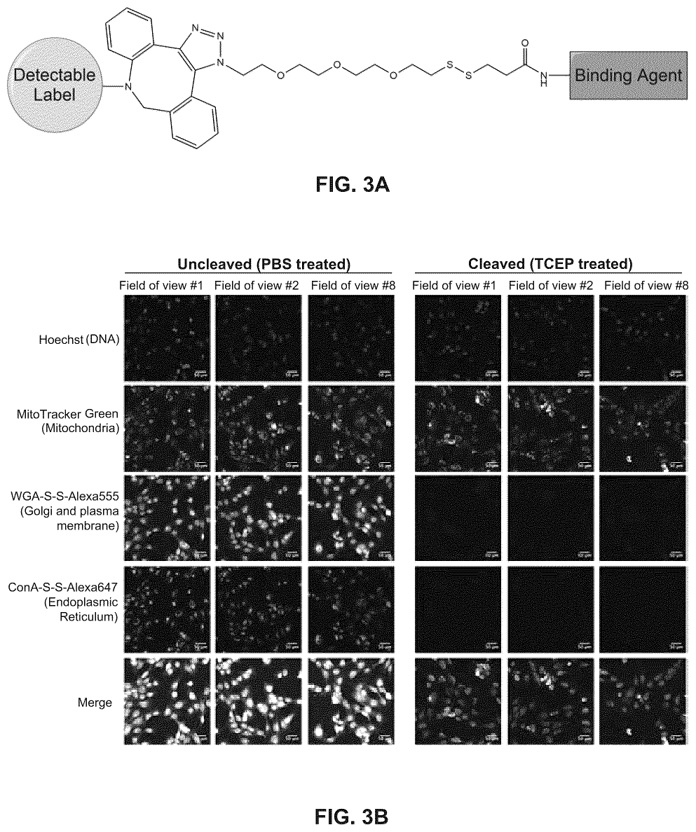

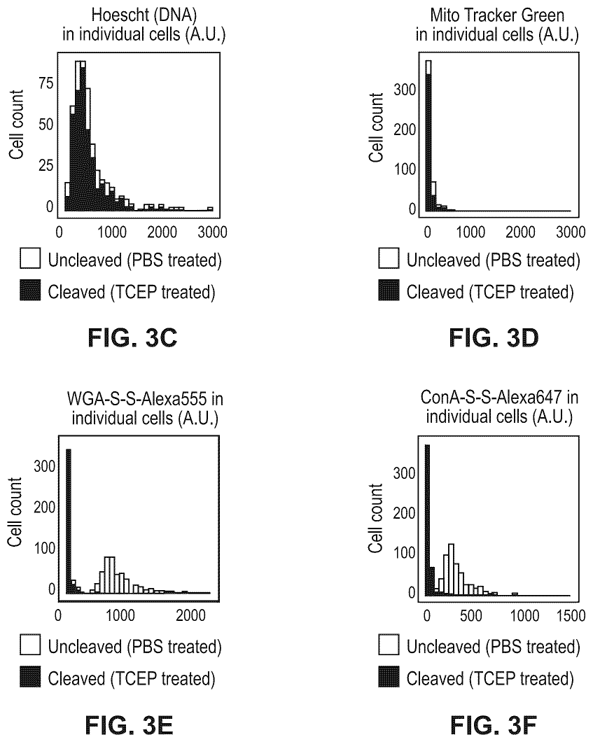

In some embodiments, the probe comprises the structure set forth in FIG. 3A. In certain embodiments, the probe comprises a linker having the linker structure set forth in FIG. 3A. In some embodiments, the binding agent of the probe comprises a lectin, optionally wherein the lectin is selected from ConA and WGA. In some embodiments, the detectable label of the probe comprises a fluorophore, e.g., wherein the fluorophore is an Alexa Fluor fluorophore. In some embodiments, the detectable label of the probe comprises Alexa Fluor 555 or Alexa Fluor 647. In some embodiments, the probe comprises ConA and Alexa Fluor 647 covalently attached by a disulfide linker. In some embodiments, the probe comprises WGA and Alexa Fluor 555 covalently attached by a disulfide linker.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in certain embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1: Production of Probes Conjugated to Dyes with Cleavable Linkers

This Example describes the production and testing of probes conjugated to fluorescent dyes with a chemically-cleavable linker. The feasibility of this approach was tested by conjugating chemically cleavable disulfide linkers to two lectin-based probes: Wheat Germ Agglutinin (WGA) and Concanavilin A (ConA) (FIG. 3A). WGA can be used to detect cell membranes and the Golgi body, and ConA can be used to detect the endoplasmic reticulum (ER).

To test the ability of the probes to specifically label analytes, AC16 cells were grown on glass coverslips were incubated with 200 nM MitoTracker Green for 30 minutes and subsequently fixed by addition of 4% formaldehyde for 20 minutes. Cells were washed in 1×HBSS, permeabilized with HBSS+0.1% Triton for 15 minutes, and washed again. Permeabilized cells were stained for 30 minutes in 1×HBSS, 5 μg/mL Hoescht, 1.5 μg/mL WGA-Disulfide-Alexa555, and 100 μg/mL Concanavilin A-Disulfide-Alexa647. Stained cells were washed three times in 1×HBSS and once in 1×PBS. Buffer was then aspirated out and replaced with either PBS alone (as a control) or PBS+50 mM TCEP (a reducing agent, Tris(2-carboxyethyl) phosphine hydrochloride, Sigma C4706) for 15 minutes. Cells were then washed three times in 1×PBS, mounted onto coverslips and imaged.

Imaging was performed on a Zeiss LSM 880+AiryScan with a 20× air objective, using the same excitation and emission parameters for all samples and all fields of view. Excitation and emission filters were set to maximize signal from the relevant dye while excluding signal from other dyes. Eight and ten fields-of-view were imaged for PBS and PBS+50 mM TCEP, respectively, with all fields-of-view selected without examining any channel except DNA (Hoescht) stain. Images were processed identically in Image J (Fiji). (FIG. 3B).

These results were computationally analyzed by segmenting individual cells and measuring the intensity of each channel per cell in the un-cleaved vs. cleaved conditions (n=465 cell and 407 cells in the un-cleaved vs. cleaved conditions, respectively) Quantification was done using custom python scripts. Nuclei were identified using Hoescht staining and cell boundaries were called using MitoTracker Green, and the signal in each channel was then quantified independently. Analysis was performed on 8 fields of view (totaling >400 cells) per condition. A two-sided Wilcoxon rank sum test was used to test for a statistically significant difference between the intensity values in un-cleaved vs. cleaved (PBS vs. TCEP) (FIGS. 3C-3F). WGA-S-S-Alexa555 signal was reduced >10-fold and the ConA-S-S-Alexa647 was reduced >5-fold upon fluorophore cleavage (FIGS. 3E and 3F). Thus, use of cleavable linkers made these fluorescent channels available again for downstream imaging. Table 1 summarizes the results shown in FIGS. 3C-3F in Arbitrary Units (A.U.).

TABLE 1
Quantification of Median Fluorescence of Label Intensity
per Cell with and without Probe Cleavage

| | Hoechst | MitoTracker | WGA | ConA |
|---|---|---|---|---|
| Uncleaved (PBS) | 506 | 109 | 798 | 348 |
| Cleaved (TCEP) | 513 | 93 | 68 | 62 |

### Example 2: Employment of Probes with Cleavable Linkers for Visualization of Multiple Analytes in a Single Channel

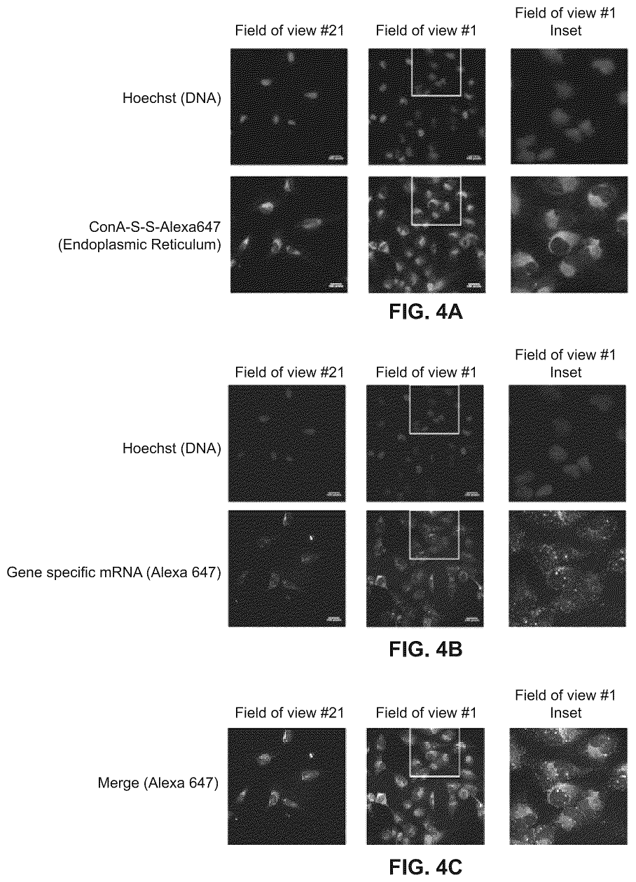

This Example was designed to demonstrate that the use of fluorescent dyes tagged to probes via cleavable linkers works effectively for multiplexed imaging through multiple cycles. Fixed AC16 cells were stained with Hoescht and ConA-S-S-Alexa647 and imaged, and the Alexa647 was subsequently chemically cleaved (using the reducing agent TCEP in this case). The cells were then re-stained to detect a gene specific mRNA (FLNC), in this case using hybridization chain reaction with FLNC specific probes that initiate a signal also in Alexa647. The nuclei from these two round of imaging were aligned to one another to ensure that the signal from each round of Alexa647 imaging was coming from the same cells. (FIGS. 4A and 4B). ConA-S-S-Alexa Fluor 647 was chosen due to its weaker cleavage ability than the WGA conjugate tested in Example 1, and thus its functionality for re-imaging in the same channel is a more stringent test. The same cells that were imaged in the first round were reimaged and merged images were generated (FIG. 4B). The merged images shown in FIG. 4C show that the signal for ConA-S-S-Alexa647 (detecting the Endoplasmic Reticulum) and the gene-specific mRNA in Alexa647 do not overlap, despite being imaged using the same fluorophore. These results indicate that this method can reliably allow for imaging multiple analytes in the same channel.

### Example 3: Employment of Probes with Cleavable Linkers for Visualization of Analytes in Tissue Samples

This Example was designed to demonstrate that the use of fluorescent dyes conjugated to probes via cleavable linkers works effectively for imaging in tissue samples.

A mouse FFPE tumor sample was obtained from a human patient-derived xenograft (5 μm thick) and prepared for staining and imaging. Briefly, the tumor sample was deparaffinized by baking at 60° C. for 2 hours and then washed three times in Xylene (3 minutes per wash). The tumor sample was rehydrated by washing twice in 100% ethanol, once in 95% ethanol, once in 70% in ethanol, once in 50% ethanol, three times in water, and twice in PBS (3 minutes per each wash). The rehydrated sample was then taken through antigen retrieval by steaming at 95° C. for 40 minutes in 1× citrate buffer (pH 6.0) and slow cooling to room temperature for 20 minutes.

After washing in PBS, samples were stained for 30 minutes at room temperature in 1×PBS+1% BSA+0.1% triton containing: 1.5 g/mL WGA-S-S-Alexa555 (1.5 μg/mL), ConA-S-S-Alexa647 (5 μg/mL), and Hoechst (10 μg/mL). Samples were washed three time in 1×PBS and then imaged using an Operetta CLS with a 20× air objective (first round of imaging). After imaging, the sample was washed in PBS, and subsequently washed three times for 10 minutes each in PBS supplemented with the reducing agent 50 mM TCEP (50 mM). Samples were washed three times in PBS again before being re-imaged using the same imaging conditions as used in the first round of imaging. In both rounds of imaging, over 100 fields-of-view were taken. Representative fields-of-view shown were processed identically using Image J (FiJi).

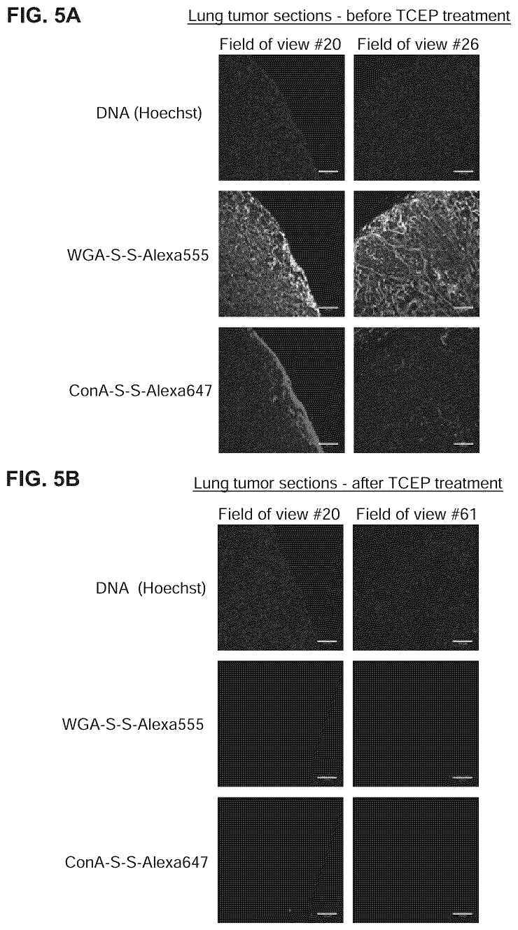

Representative micrographs of the tumor sample stained with WGA-S-S-Alexa555, ConA-S-S-Alexa647, and Hoescht prior to treatment with the reducing agent are shown in FIG. 5A, and micrographs of the same tumor sample following treatment with the reducing agent are shown in FIG. 5B. Both WGA-S-S-Alexa555 and ConA-S-S-Alexa647 signal were efficiently removed by treatment with the reducing agent TCEP, allowing the fluorescent channels to be used again in downstream imaging. These results indicate that the method and probes can reliably be used to image tissue samples.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for detecting the presence or absence, amount, location, morphology and/or spatial patterning of at least a first and a second analyte in a sample, the method comprising:
(i) contacting the sample with a first probe comprising (a) a first binding agent capable of binding to the first analyte, and (b) a first detectable label, wherein the first binding agent and the first detectable label are conjugated by a first cleavable linker, under conditions to permit the first probe to bind the first analyte if the first analyte is present in the sample;
(ii) detecting the first detectable label which, if present, is indicative of the presence or absence, amount, location, morphology and/or spatial patterning of the first analyte in the sample;
(iii) cleaving the first cleavable linker in the first probe, thereby removing the first detectable label from the sample;
(iv) contacting the sample with a second probe comprising (a) a second binding agent capable of binding to the second analyte, and (b) a second detectable label, optionally wherein the second binding agent and the second detectable label are conjugated by a second cleavable linker, under conditions to permit the second probe to bind the second analyte if the second analyte is present in the sample; and
(v) detecting the second detectable label which, if present, is indicative of the presence or absence, amount, location, morphology and/or spatial patterning of the second analyte in the sample.

2. The method of claim 1, wherein the first cleavable linker and the second cleavable linker are each independently selected from the group consisting of a chemically cleavable linker, a photo-cleavable linker, and an enzymatically cleavable linker.

3. The method of claim 1 or claim 2, wherein the first cleavable linker or the second cleavable linker is a chemically cleavable linker, or the first cleavable linker and the second cleavable linker are both a chemically cleavable linker.

4. The method of any one of claims 1-3, wherein the first cleavable linker or the second cleavable comprises a disulfide bond, or the first cleavable linker and the second cleavable linker comprise a disulfide bond.

5. The method of any one of claims 1-4, wherein step (iii) comprises cleaving the first cleavable linker by contacting the sample with a first reducing agent.

6. The method of claim 5, wherein the first reducing agent is selected from the group consisting of dithiothreitol (DTT), tris(2-carboxyethyl) phosphine (TCEP), and β-mercaptoethanol (BME).

7. The method of any one of claims 1-6, wherein the first and second detectable labels are the same.

8. The method of any one of claims 1-7, wherein the method detects the presence or absence, amount, location, morphology and/or spatial patterning of at least 50 analytes, at least 40 analytes, at least 30 analytes, at least 20 analytes, at least 15 analytes, at least 10 analytes, at least 9 analytes, at least 8 analytes, at least 7 analytes, at least 6 analytes, at least 5 analytes, at least 4 analytes, at least 3 analytes, or at least 2 analytes.

9. The method of any one of claims 1-8, wherein the first or second binding agent is a protein.

10. The method of any one of claims 1-9, wherein the first or second binding agent is a lectin.

11. The method of any one of claims 1-10, wherein the first or second binding agent is Concanavalin A (ConA), wheat germ agglutinin (WGA), Isolectin GS-IB4, Lectin GS-II, or PNA lectin.

12. The method of any one of claims 1-11, wherein the first or second binding agent is ConA.

13. The method of any one of claims 1-11, wherein the first or second binding agent is WGA.

14. The method of any one of claims 1-8, wherein the first or second binding agent is a small molecule.

15. The method of any one of claims 1-14, wherein the first or second binding agent is not a nucleic acid.

16. The method of any one of claims 1-15, wherein the first and second detectable labels produce the same or a similar detectable signal.

17. The method of any one of claims 1-16, wherein the first or second detectable label is a fluorophore.

18. The method of claim 17, wherein the fluorophore is selected from the group consisting of an Alexa Fluor fluorophore, an ATTO dye, a cyanine dye, fluorescein, a fluorescent protein (e.g., GFP), a rhodamine dye, and a quenching agent.

19. The method of claim 18, wherein the fluorophore is selected from Alexa Fluor 488, Alexa Fluor 555, Alexa Fluor 568, Alexa Fluor 594, Alexa Fluor 633, Alexa Fluor 647, and Alexa Fluor 750.

20. The method of any one of claims 1-19, wherein the first and second detectable labels are fluorophores that have substantially overlapping excitation and/or emission spectra.

21. The method of any one of claims 1-20, wherein step (i) comprises contacting the sample with at least 2 probes, at least 3 probes, at least 4 probes, at least 5 probes, at least 6 probes, at least 7 probes, at least 8 probes, at least 9 probes, or at least 10 probes.

22. The method of any one of claims 1-21, wherein step (iv) comprises contacting the sample with at least 2 probes, at least 3 probes, at least 4 probes, at least 5 probes, at least 6 probes, at least 7 probes, at least 8 probes, at least 9 probes, or at least 10 probes.

23. The method of any one of claims 1-22, wherein the method further comprises:
(vi) cleaving the second cleavable linker in the probe in contact with the sample, thereby removing the second detectable label from the sample.

24. The method of claim 23, wherein step (vi) comprises cleaving the second cleavable linker by contacting the sample with a second reducing agent.

25. The method of claim 24, wherein the second reducing agent is selected from the group consisting of DTT, TCEP, and BME.

26. The method of claim 24 or claim 25, wherein the first reducing agent and the second reducing agent are the same.

27. The method of any one of claims 23-26, wherein the method further comprises:
(vii) contacting the sample with a further probe comprising (a) a further binding agent capable of binding to a further analyte, and (b) a further detectable label, optionally wherein the further binding agent and the further detectable label are conjugated by a further cleavable linker, under conditions to permit the further probe to bind the further analyte if the further analyte is present in the sample; and
(viii) detecting the further detectable label which, if present, is indicative of the presence, amount, location, morphology and/or spatial patterning of the further analyte in the sample.

28. The method of claim 27, wherein the method further comprises one or more iterations of:
(ix) cleaving the cleavable linker in the probe in contact with the sample, thereby removing the detectable label from the sample, and
subsequently repeating step (vii) and step (viii).

29. The method of claim 28, wherein the method comprises 2 or more, 3 or more, 4 or more, 5 or more, 6 or more, 7 or more, 8 or more, 9 or more, 10 or more, 11 or more, 12 or more, 13 or more, 14 or more, or 15 or more iterations of steps (ix), (vii), and (viii).

30. The method of any one of claims 27-29, wherein the further cleavable linker is selected from the group consisting of a chemically cleavable linker, a photo-cleavable linker, and an enzymatically cleavable linker.

31. The method of any one of claims 27-30, wherein the further cleavable linker comprises a disulfide bond.

32. The method of claim 30 or claim 31, wherein step (ix) comprises cleaving the chemical linker by contacting the sample with a further reducing agent.

33. The method of claim 32, wherein the further reducing agent is selected from the group consisting of DTT, TCEP, and BME.

34. The method of claim 32 or claim 33, wherein the first, second, and further reducing agents are the same.

35. The method of any one of claims 27-34, wherein the first, second, and further detectable labels are the same.

36. The method of any one of claims 27-35, wherein the further probe in each of the iterations of steps (ix), (vii), and (viii) comprises a different binding agent.

37. The method of any one of claims 27-36, wherein the further binding agent is a protein.

38. The method of any one of claims 27-37, wherein the further binding agent is a lectin.

39. The method of any one of claims 27-38, wherein the further binding agent is Concanavalin A (ConA), wheat germ agglutinin (WGA), Isolectin GS-IB4, Lectin GS-II, or PNA lectin.

40. The method of any one of claims 27-39, wherein the further binding agent is ConA.

41. The method of any one of claims 27-39, wherein the further binding agent is WGA.

42. The method of any one of claims 27-36, wherein the further binding agent is a small molecule.

43. The method of any one of claims 27-42, wherein the further binding agent is not a nucleic acid.

44. The method of any one of claims 27-43, wherein the first, second, and further detectable labels produce the same or a similar detectable signal.

45. The method of any one of claims 27-44, wherein the further detectable label is a fluorophore.

46. The method of claim 45, wherein the further detectable label is selected from the group consisting of an Alexa Fluor fluorophore, an ATTO dye, a cyanine dye, fluorescein, a fluorescent protein (e.g., GFP), a rhodamine dye, and a quenching agent.

47. The method of claim 45 or 46, wherein the further detectable label is selected from Alexa Fluor 488, Alexa Fluor 555, Alexa Fluor 568, Alexa Fluor 594, Alexa Fluor 633, Alexa Fluor 647, and Alexa Fluor 750.

48. The method of any one of claims 27-47, wherein the first, second, and further detectable labels are fluorophores that have substantially overlapping excitation and/or emission spectra.

49. The method of any one of claims 27-48, wherein step (vii) comprises contacting the sample with at least 2 probes, at least 3 probes, at least 4 probes, at least 5 probes, at least 6 probes, at least 7 probes, at least 8 probes, at least 9 probes, or at least 10 probes.

50. The method of any one of claims 1-49, wherein the sample is selected from a tissue sample, a liquid sample, and a cell sample.

51. The method of claim 50, wherein the sample is a cell sample selected from a two-dimensional cell culture sample, a three-dimensional cell culture sample, a suspension cell culture sample, an organoid sample, a heterogeneous cell culture sample, and a patient-derived cell sample.

52. The method of any one of claims 1-51, wherein the first, second, and further analytes are each independently selected from the group consisting of a cell, organelle, protein, peptide, carbohydrate, glycoprotein, glycopeptide, glycolipid, lipid, lipoprotein, nucleic acid, and nucleoprotein.

53. The method of claim 52, wherein the first, second, and further analytes are each independently selected from the group consisting of an endoplasmic reticulum, a plasma membrane, a Golgi body, a microtubule, an actin filament, a sarcomere, and a collagen fibril.

54. A method for detecting the presence or absence, amount, location, morphology and/or spatial patterning of at least a first and a second analyte in a sample, wherein the first analyte is the endoplasmic reticulum (ER) and/or a protein, carbohydrate, glycoprotein, or glycolipid enriched in the ER, the method comprising:
(i) contacting the sample with a first probe comprising (a) ConA, and (b) a first fluorophore, wherein ConA and the first fluorophore are conjugated by a first cleavable linker comprising a disulfide bond, under conditions to permit ConA to bind the ER and/or the protein, carbohydrate, glycoprotein, or glycolipid enriched in the ER;
(ii) detecting the first fluorophore;
(iii) cleaving the disulfide bond in the first probe by addition of a reducing agent, thereby removing the first fluorophore from the sample;
(iv) contacting the sample with a second probe comprising (a) a binding agent capable of binding to the second analyte, and (b) a second fluorophore, optionally wherein the binding agent and the second fluorophore are conjugated by a second cleavable linker comprising a disulfide bond; and
(v) detecting the second fluorophore which, if present, is indicative of the presence, amount, location, morphology and/or spatial patterning of the second analyte in the sample.

55. A method for detecting the presence or absence, amount, location, morphology and/or spatial patterning of at least a first, a second, and a third analyte in a sample, wherein the first analyte is a plasma membrane and/or a protein, carbohydrate, glycoprotein, or glycolipid enriched in the plasma membrane and the second analyte is a Golgi body and/or a protein, carbohydrate, glycoprotein, or glycolipid enriched in the Golgi body, the method comprising:
(i) contacting the sample with a first probe comprising (a) WGA, and (b) a first fluorophore, wherein WGA and the first fluorophore are conjugated by a first cleavable linker comprising a disulfide bond, under conditions to permit WGA to bind the plasma membrane and/or the protein, carbohydrate, glycoprotein, or glycolipid enriched in the plasma membrane and the Golgi body and/or the protein, carbohydrate, glycoprotein, or glycolipid enriched in the Golgi body;
(ii) detecting the first fluorophore;
(iii) cleaving the disulfide bond in the first probe by addition of a reducing agent, thereby removing the first fluorophore from the sample;
(iv) contacting the sample with a second probe comprising (a) a binding agent capable of binding to the third analyte, and (b) a second fluorophore, optionally wherein the binding agent and the second fluorophore are conjugated by a second cleavable linker comprising a disulfide bond; and
(v) detecting the second fluorophore which, if present, is indicative of the presence or absence, amount, location, morphology and/or spatial patterning of the third analyte in the sample.

56. The method of claim 54 or claim 55, wherein the reducing agent is selected from the group consisting of DTT, TCEP, and BME.

57. The method of any one of claims 54-56, wherein the binding agent is a protein.

58. The method of any one of claims 54-57, wherein the binding agent is a lectin.

59. The method of claim 54, wherein the binding agent is WGA.

60. The method of claim 55, wherein the binding agent is ConA.

61. The method of any one of claims 54-56, wherein the binding agent is a small molecule.

62. The method of any one of claims 54-61, wherein the binding agent is not a nucleic acid.

63. The method of any one of claims 54-62, wherein the first and second fluorophores produce the same or a similar detectable signal.

64. The method of any one of claims 54-63, wherein the first and second fluorophores are independently selected from the group consisting of an Alexa Fluor fluorophore, an ATTO dye, a cyanine dye, fluorescein, a fluorescent protein (e.g., GFP), a rhodamine dye, and a quenching agent.

65. The method of any one of claims 54-64, wherein the first and second fluorophores are independently selected from the group consisting of Alexa Fluor 488, Alexa Fluor 555, Alexa Fluor 568, Alexa Fluor 594, Alexa Fluor 633, Alexa Fluor 647, and Alexa Fluor 750.

66. The method of any one of claims 54-65, wherein the first and second fluorophores have substantially overlapping excitation and/or emission spectra.

67. The method of any one of claims 54-66, wherein the method further comprises:
(vi) cleaving the second cleavable linker in the probe in contact with the sample, thereby removing the second fluorophore from the sample.

68. The method of claim 67, wherein step (vi) comprises cleaving the second cleavable linker by contacting the sample with a second reducing agent.

69. The method of claim 68, wherein the second reducing agent is selected from the group consisting of DTT, TCEP, and BME.

70. A probe comprising a binding agent capable of binding to an analyte of interest, and a detectable label, wherein the binding agent and the detectable label are conjugated by a cleavable linker comprising a disulfide bond.

71. The probe of claim 70, wherein the binding agent is a protein.

72. The probe of claim 70 or claim 71, wherein the binding agent is a lectin.

73. The probe of any one of claims 70-72, wherein the binding agent is Concanavalin A (ConA) or wheat germ agglutinin (WGA).

74. The probe of claim 70, wherein the binding agent is a small molecule.

75. The probe of any one of claims 70-74, wherein the detectable label is a fluorophore.

76. The probe of claim 75, wherein the fluorophore is selected from an Alexa fluorophore, an ATTO dye, a cyanine dye, fluorescein, a fluorescent protein (e.g., GFP), a rhodamine dye, and a quenching agent.

77. The probe of claim 76, wherein the fluorophore is selected from Alexa Fluor 488, Alexa 5 Fluor 555, Alexa Fluor 568, Alexa Fluor 595, Alexa Fluor 633, Alexa Fluor 647, and Alexa Fluor 750.

78. A probe comprising ConA and a fluorophore, wherein ConA and the fluorophore are conjugated by a cleavable linker comprising a disulfide bond.
